(19) United States
(12) Patent Application Publication
(10) Pub. No.: US 2024/0226882 A1
(43) Pub. Date: Jul. 11, 2024

(54) Plasma Membrane on a Chip

(71) Applicant: Yale University, New Haven, CT (US)

(72) Inventors: Sathish Ramakrishnan, New Haven, CT (US); Ramalingam Venkat Kalyana Sundaram, New Haven, CT (US); Manindra Bera, New Haven, CT (US)

(21) Appl. No.: 18/409,011

(22) Filed: Jan. 10, 2024

Related U.S. Application Data
(60) Provisional application No. 63/479,426, filed on Jan. 11, 2023.

(51) Int. Cl. B01L 3/00; G01N 21/64; G01N 21/65

(52) U.S. Cl. CPC B01L 3/502715; G01N 21/6458; G01N 21/65; B01L 2300/0645; B01L 2300/0819; B01L 2300/12

(57) ABSTRACT

The present invention provides membrane on a chip devices for studying lipid membranes. The devices are suitable for studying molecular interactions on lipid membranes with single-molecule resolution. The devices are also are applicable to the study of membrane properties, interactions between membranes and vesicles, viruses, bacteria, and the like, and trafficking of compounds across the membrane, among others. The devices are readily amenable to a variety of microscopy and spectroscopy techniques.

### CROSS REFERENCE TO RELATED APPLICATIONS

This application claims priority to U.S. Provisional Patent Application No. 63/479,426, filed Jan. 11, 2023, the contents of which are incorporated by reference herein in their entirety.

### BACKGROUND OF THE INVENTION

The plasma membrane regulates material exchange between the cell and its surroundings. The structure and composition of the plasma membrane are essential for numerous cellular activities, such as protein complex formation and receptor-mediated signaling (Singer, S. J., et al., 1972, Science, 175(4023):720-731). However, obtaining structural and dynamic information on membrane protein complexes is one of the most significant challenges in cell and molecular biology. Most of the in vitro platforms to study membrane protein interactions can be grouped into two broad categories: curved or planar bilayers. Curved bilayers can be achieved with liposomes in the format of small unilamellar vesicles (SUVs), large unilamellar vesicles (LUVs), and giant unilamellar vesicles (GUVs) for studying lipid and membrane proteins due to their two-sided compartmentalization and excellent lipids and protein diffusion properties (Sessa, G., et al., 1968, Journal of Lipids Research, 9(3):310-318). However, achieving consistency in terms of size or lipid asymmetric distribution can be difficult, and circular geometry is not suitable for high-resolution microscopy or electrophysiological studies. Droplet interface bilayers (DIBs) offer several advantages like liposomes, but the bulk oil environment and the degree of incorporation of oils at the droplet interface restrict its application in biological studies (Leptihn, S., et al., 2013, Nature Protocols, 8(6):1048-1057).

Planar bilayers in the format of supported or suspended lipid bilayers have attracted considerable interest in overcoming these challenges. They provide simplified biomimetic approaches to elucidate fundamental cellular processes like exo- and endocytosis, ion transport, antibiotic and pathogenic interactions, protein organization, sequencing, and signaling (Yu, H. J., et al., 2019, Methods in Molecular Biology, 1860:237-249; Nimigean, C. M., et al., 2006, Nature Protocols, 1(3):1207-1212; Lim, J. H., et al., 2015, ACS Nano, 9(2): 1699-1706; Rao, L., et al., 2020, ACS Nano, 14(3): 2569-2574; Pinheiro, M., et al., 2019, Chemistry and Physics of Lipids, 222:36-46; Su, X. L., et al., 2016, Science, 352(6285):595-599; Derrington, I. M., et al., 2010, Proceedings of the National Academy of Sciences USA, 107(37). 16060-16065, Elbaum-Garfinkle, S., et al., 2010, Biophysical Journal, 98(11):2722-2730). Most in vitro studies happen on suspended or supported bilayers using reconstituted proteins. The common form of suspended bilayers, black lipid membranes (BLMs), made with an organic solvent may lead to non-physiological results, and the setups used are also incompatible for single-molecule and high-resolution imaging (Ogishi, K., et al., 2022, Lab on a Chip, 22(5): 890-898; Kamiya, K., et al., 2021, Sensors and Actuators B: Chemical, 128917). BLMs are commonly created by a painting method (depositing lipids dissolved in decane) in micro-apertures fabricated in glass pipettes, Teflon films, or silicon nitride (White, R. J., et al., 2007, Journal of the American Chemical Society, 129(38): 11766-11775; Bright, L. K., et al., 2015, ACS Biomaterials Science and Engineering, 1(10):955-963; Bartsch, P., et al., 2012, Materials, 5(12):2705-2730; Borisenko, V., et al., 2003, Biophysical Journal, 84(1):612-622; Han, X. J., et al., 2007, Advanced Materials, 19(24):4466-4470). Forming BLMs requires a high degree of skill, and yet the outcome of the bilayer is short-lived and unstable, which makes it unsuitable for monitoring dynamic biological processes (Mueller, P., et al., 1962, Nature, 194(4832):979-980; Miller, C., 2013, Springer Science & Business Media).

The alternative approach is to use supported bilayers, spread on substrates like glass, mica, or Si/SiO₂ to determine structural and dynamic information of macromolecular complexes (Cremer, P. S., et al., 1999, Journal of Physical Chemistry B, 103(13): 2554-2559; Han, X., et al., 2007, Chemistry, 13(28):7957-7964; Danelon, C., et al., 2006, Langmuir, 22(1):22-25). However, their applications in protein studies are limited due to the lack of an aqueous compartment on the support side of the bilayer, resulting in limited space (˜10-20 Å) between the lipid headgroups and the surface thereby reducing lipid and protein diffusion. It is also difficult to reconstitute bulky, multi-pass transmembrane domain-containing proteins while controlling lipid and protein orientation. Finally, most lipids have asymmetric distributions between the leaflets of cellular bilayer which are very difficult to recapture in reconstituted systems (Chiang, P.-C., et al., 2017, Scientific Reports, 7(1) 15139; Op dem Kamp, J. A., et al., 1979, Annual Review of Biochemistry, 48:47-71). Proteins such as ion channels also have a directionality when embedded within bilayers to regulate the flow in and out of the cells. Reconstitutions result in the symmetrical orientation of lipids and proteins resulting in about half the proteins not participating in the reaction due to incorrect directionality. Therefore, methods to achieve native lipid and protein asymmetry are needed to elucidate the roles of proteins in complex biological processes.

As such, there is a need in the art for devices and methods for examining the dynamics of fluid membrane bilayers with high precision in a native environment. The present invention addresses this long felt, but unmet, need.

### SUMMARY OF THE INVENTION

In one aspect, the present invention relates to a membrane on a chip device comprising: a bottom layer of an essentially impermeable substrate having a first thickness; a chip layer of an essentially impermeable substrate having a second thickness stacked on top of the bottom layer; and a barrier layer of an essentially impermeable material having a third thickness stacked on top of the chip layer; wherein the barrier layer comprises one or more macro-holes extending through the thickness of the barrier layer with a first diameter, exposing the chip layer; and wherein the chip layer comprises a plurality of holes extending through the thickness of the barrier layer with a second diameter, wherein the plurality of holes is arranged in a number of rows and columns with a regular distance separating the holes of each row and each column.

In some embodiments, the first layer comprises SF11 glass, soda-lime glass, borosilicate glass, aluminosilicate glass, or glass-ceramic. In some embodiments, the first layer has a thickness of about 0.05 mm to about 5 mm. In some embodiments, the thickness is about 150 μm.

In some embodiments, the second layer comprises Si/SiO₂ or polydimethylsiloxane (PDMS) and has a thickness of about 2.5 μm to about 6 μm.

In some embodiments, the holes are cylindrical and have a diameter of about 1-7.5 μm. In some embodiments, the holes are right circular frustra with a lower diameter greater than the upper diameter, wherein the upper diameter is about 1 μm to about 10 μm and the lower diameter is about 1.1-15.4 μm. In some embodiments, the distance between holes in the rows and the distance between holes in the columns is about half the diameter of the holes.

In some embodiments, the thickness of the second layer is 4.7 μm, the holes are cylindrical, the diameter of the holes is 5 μm, and the distance between the holes is 2.5 μm.

In some embodiments, the thickness of the second layer is 4.7 μm, the holes are frustra with an upper diameter of about 5 μm and a lower diameter of about 14.1 μm, and the distance between the holes is 5 μm.

In some embodiments, the holes of a row are connected by a series of channels. In some embodiments, the first and last holes are connected to openings on the exterior edge of the second layer by channels such that a fluid could flow from the opening connected to the first hole, through the channels and holes, out the opening connected to the last hole.

In some embodiments, the holes comprise electrodes operably linked to wires which extend through the first layer of the chip.

In another aspect, the present invention is related to a system for studying membranes comprising a chip of the present invention and a microscope, spectrometer, or spectrophotometer. In some embodiments, the microscope is a total internal fluorescence (TIRF) microscope. In some embodiments, the spectrometer is a Raman spectrometer.

In some embodiments of the system, the membrane contains proteins with specific directionality and symmetric or asymmetric distribution of lipids between leaflets. In some embodiments, the membranes are reconstituted. In some embodiments, the membranes are harvested from cells or parts of live or fixed cells. In some embodiments, the membrane is suspended in the system such that it is compatible with single-molecule studies.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1D, depicts representative images of the tunable suspended lipid membrane (SLIM) platform design and characterization. FIG. 1A depicts a schematic representation of the principle of the SLIM platform on a tunable silicon microarray chip. The fluorophores in the lipid bilayer are visualized by TIRF microscopy using an index-matching medium between the glass and buffer solution (n₁=1.52, n₃=1.33). Inset: By tuning the refractive index of the media between the bilayer and the glass (n₂=1.41), total internal reflection occurs at the bilayer, 4.7 μm above the glass. FIG. 1B depicts a schematic representation of the silicon chip, which contains 5 μm holes, allowing an inverted objective to visualize events occurring on the bilayer. GUVs reconstituted with proteins are burst on this grid to create unilamellar bilayers. FIG. 1C depicts a representative fluorescent image of unilamellar bilayers produced from burst GUVs. Scale bar=5 μm. FIG. 1D depicts representative FRAP measurements of lipids containing 2 mol % NBD-DOPE or reconstituted, pre-assembled t-SNARE proteins (SNAP25-Q20C) labeled with AlexaFluor 488-maleimide in conditions with buffer (orange) or OptiPrep™ (green). The lipids diffuse at 3.2±0.9 μm²/sec in buffer and 2.8±0.3 μm²/sec in OptiPrep™. The proteins diffuse at 1.6±0.6 μm²/sec in buffer and 1.8±0.5 μm²/sec in OptiPrep™.

FIG. 2, comprising FIG. 2A through FIG. 2D, depicts representative images of SLIM platform fabrication. FIG. 2A depicts a representative image of the silicon chip fabricated using shadow mask technology. Holes with 5 μm diameter are etched 5 μm apart into the 5 μm silicon chip which is then adhered to cover glass. The base of the platform is built on a plastic support with an adhesive backing (LSE300, 3M) that holds the silicon chip in place. An iBidi sticky slide is attached on top to create a chamber. FIG. 2B depicts a representative schematic of the silicon surface, which can be modified to enhance lipid binding. This is typically done using potassium hydroxide or plasma treatment, which increases surface charge. This allows for binding with charged lipids. FIG. 2C depicts a representative SEM image of the fabricated chip. Scale bar=10 μm. FIG. 2D depicts representative characterization of the lipid diffusion using different chip heights.

FIG. 3, comprising FIG. 3A and FIG. 3B, depicts representative TIRF evanescent wave penetration depth as a function of refractive index, calculated by the formula:

$$d = \frac{{λ}_{o}}{4 π \sqrt{{n}_{OptiPrep}^{2} sin {θ}^{2} - {n}_{water}^{2}}}$$

FIG. 3A depicts representative penetration depth in a classical TIRF setup using 488 nm and 647 nm excitation wavelengths. FIG. 3B depicts representative penetration depth in the SLIM platform using 488 nm and 647 nm excitation wavelengths.

FIG. 4, comprising FIG. 4A through FIG. 4D, depicts representative characterization of the OptiPrep™ index-matching buffer. FIG. 4A depicts representative measurement of the refractive index of distilled water vs. buffer with increasing amounts of OptiPrep™. FIG. 4B depicts representative emission spectra measurements of buffer with 0% and 45% OptiPrep™. FIG. 4C depicts representative imaging of Tetraspeck™ beads (100 nm) at 488 nm and 647 nm under buffer and index-matching conditions (45% OptiPrep™). FIG. 4D depicts representative line profiles across the beads obtained for the three different sized beads. The full width half maxima were 100 nm_{buffer}: 0.54 μm, 100 nm_{OptiPrep}: 0.58 μm, 500 nm_{buffer}: 0.78 μm, 500 nm_{OptiPrep}: 0.71 μm, 1 μm_{buffer}: 1.59 μm, 1 μm_{OptiPrep}: 1.57 μm. Images were recorded with a 60×/1.49 oil immersion objective. Scale bar=5 μm.

FIG. 5, comprising FIG. 5A through FIG. 5E, depicts representative characterization of reconstituted suspended membranes. FIG. 5A depicts a representative fluorescence recovery trace from lipid diffusion measurements (FRAP) and a panel of representative images showing lipid fluorescence (NBD-DOPE) recovery at selected times. FIG. 5B depicts a representative protein fluorescence bleaching trace used to measure protein diffusion and representative pre- and post-bleach images along with the bleach steps. Pre-assembled t-SNAREs with a single cysteine (SNAP25-Q20C) labeled with AlexaFluor 647 were used to determine the rate of protein diffusion. FIG. 5C depicts representative images of reconstituted bilayers created on a chip, which were found to last over 3 hours. FIG. 5D depicts the fluorescence intensity measured from images as depicted in FIG. 5C over time, suggesting the bilayer was stable for over 3 hours. FIG. 5E depicts representative bilayer bending measured on the SLIM platform in buffer with and without 45% OptiPrep. For comparison, an older generation chip (Confocal) was also tested.²

FIG. 6, comprising FIG. 6A through FIG. 6C, depicts representative characterization of bilayer integrity and lamellarity. FIG. 6A depicts a schematic representation and representative imaging of bilayer integrity tested by trapping sulforhodamine B (SRB, 20 mM) within the holes below the bilayer. While the bilayer (top, green—NBD-DOPE) is present, the SRB (red) remains trapped. When the bilayer is dissolved with the addition of detergent (10% SDS, bottom), the dye is released, and the signal within the hole is lost. Scale bar=5 μm. FIG. 6B depicts a schematic representation and results for a transport assay using pore-forming toxin α-hemolysin used to validate the formation of a unilamellar lipid bilayer using reconstituted vesicles. FIG. 6B depicts a schematic representation and results for a transport assay using pore-forming toxin α-hemolysin used to validate the formation of a unilamellar lipid bilayer using cell-derived vesicles. For both FIGS. 6A and 6B, calcium (25 mM) was trapped below the bilayer while Calcium Green™ was added above. Addition of α-Hemolysin results in a fluorescence increase which depicted to the right.

FIG. 7, comprising FIG. 7A through FIG. 7D, depicts representative single-molecule investigations on the reconstituted suspended bilayers. FIG. 7A depicts a schematic representation of a DNA scaffold with one or two fluorophores (AlexaFluor 488) attached to the bilayer through biotin-streptavidin interactions. Representative bleach traces show single or double bleaching events. FIG. 7B depicts representative results of vesicles loaded with three Synaptotagmin molecules labeled with AlexaFluor 647 to a lone cysteine residue (E269C). The vesicles were docked on bilayers containing PIP2. Scale bar=1 μm. Representative bleach traces are depicted from vesicles showing up to 3 bleach steps and a histogram of the distribution of Syt1 count per vesicle (n=79). FIG. 7C depicts a schematic representation of and results from experiments with double-stranded DNA with FRET probes Cy3 and Cy5 anchored to the bilayer using biotin-streptavidin interactions. The recorded donor and FRET emission traces are shown in green and orange respectively. FIG. 7D depicts a schematic representation of and results from protein-based FRET measured between neuronal t-SNAREs labeled with AlexaFluor 568 maleimide (SNAP25-Q20C) and v-SNARE labeled with AlexaFluor 647 maleimide (VAMP2-S28C). The recorded donor and FRET emission traces are shown in green and orange respectively.

FIG. 8, comprising FIG. 8A and FIG. 8B, depicts representative counting of the copy number of SYT1 in reconstituted vesicles. FIG. 8A depicts a representative image of a fluorescent gel (Typhoon™) of SYT1 labeled with Alexa647 floated up in vesicles. Samples treated with chymotrypsin have no SYT1 on the outside of the vesicle. The gel has been quantified in the table on the right. FIG. 8B depicts representative bleach traces of vesicles loaded with 2 and 5 copies of SYT1, as well as vesicles treated with chymotrypsin. Inset histograms depict the number of Syt1 molecules per vesicle based on bleach steps. After chymotrypsin with two copies, n=124; before chymotrypsin with five copies, n=337; after chymotrypsin with five copies, n=249.

FIG. 9, comprising FIG. 9A through FIG. 9F, depicts representative imaging and characterization of functional bilayers created from native cellular membranes. FIG. 9A depicts a schematic representation of the steps involved, including treating cells with N-ethyl maleimide (NEM), collecting the pinched-off GPMVs, and bursting them on a silicon grid. Figure (B depicts representative imaging corresponding to the steps in FIG. 9A. For steps 1 through 3, scale bar=20 μm. Bilayers created from GPMVs were visualized through the holes on the silicon grid. Scale bar=5 μm. FIG. 9C depicts representative imaging and a representative FRAP curve generated from the bilayer to measure lipid diffusion (D=2.4 μm²/sec). FIG. 9D depicts representative imaging and a representative FRAPP curve for proteins (red) labeled with Alexa647-NHS ester to measure the diffusion coefficient (1.8 μm²/sec). FIG. 9E depicts a representative histogram showing the distribution of GPMV diameters with a mean of 15.4±4.2 μm (n=117). FIG. 9F depicts representative imaging of antibody staining for SNAP25, Syntaxin1a, and LRP1 (red) on the native bilayer (green). Scale bar=5 μm.

FIG. 10, comprising FIG. 10A though FIG. 10F, depicts representative imaging and characterization of native suspended membranes from INS-1 and EpiHEK293 cells. FIG. 10A depicts representative imaging of GPMVs produced from INS-1 cells and the resulting suspended bilayers. FIG. 10B depicts a representative fluorescence intensity trace from a z-stack of the bilayer with a FWHM of 2 μm. FIG. 10C depicts representative images of lipid diffusion on INS-1 cell-derived membranes during FRAP experiments and a representative fluorescence recovery trace. FIG. 10D depicts representative imaging of protein diffusion measurement using NHS-ester-AlexaFluor 647-labeled proteins and a representative photobleaching trace. FIG. 10E depicts representative histograms for INS-1 (green) and HEK293 (purple) GPMV diameter distributions, with an average of 9.4±4.1 μm and 8.9±4.2 μm respectively. For INS-1, n=33; for HEK293, n=125. FIG. 10F depicts representative imaging of GPMVs harvested from HEK293 suspension cells (scale bar=20 μm) labeled with MemGlow 488 and the resulting bilayers (scale bar=5 μm). Representative lipid and protein FRAP traces provided.

FIG. 11, comprising FIG. 11A and FIG. 11B, depicts representative imaging used in characterizing asymmetric organization of proteins on the native suspended membrane. FIG. 11A depicts representative images of intact cells stained for SNARE proteins Syntaxin 1a and SNAP25, found on the inner leaflet, and low-density lipoprotein-related receptor 1 (LRP1), which is found on the outer leaflet. FIG. 11B depicts representative images of GPMVs harvested from cells stained for Syntaxin1a, SNAP25, and LRP1. For FIGS. 11A and 11B, scale bar=20 μm. Antibodies were labeled with a secondary antibody labeled with AlexaFluor 647 (red) while the bilayer was labeled with MemGlow 488.

FIG. 12, comprising FIG. 12A through FIG. 12G, depicts representative imaging and characterization of the asymmetric nature of native lipid bilayer. To determine whether lipids maintain the asymmetric nature after forming planar bilayers, membranes were tested for the presence of phosphatidylserine (PS) lipids as well as raft and non-raft regions. FIG. 12A depicts a graphic representation of harvesting GPMVs and making bilayers from N2a cells while maintaining an asymmetric lipid distribution. FIG. 12B depicts a representative transmitted light image of GPMVs produced by N2a cells. Scale bar=20 μm. FIG. 12C depicts representative imaging of live N2a cells (transmitted light) stained with MemGlow 647 (green) and Annexin V (red). Scale bar=50 μm. FIG. 12D depicts a graphic representation and representative imaging of Annexin V binding exposed PS on native bilayers produced from harvested GPMVs since the inner leaflet is exposed. Annexin V staining of PS found on bilayers made from N2a GPMVs is depicted above and bilayer staining with MemGlow below. Scale bar=5 μm. FIG. 12E depicts representative Annexin V fluorescence signal increasing over time measured on the bilayer (green) and on control wells without bilayer (blue). FIG. 12F depicts representative imaging and quantification of Annexin V being removed by chymotrypsin treatment from reconstituted (symmetric) and cellular (asymmetric) bilayers. FIG. 12G depicts representative imaging of on-raft and raft domain staining as visualized by Fast DiO (green), Cholera Toxin-sub-unit-B (CT-B) (red), and the merge. FIG. 12H depicts a fluorescence trace of the line profile from FIG. 12G (white line).

FIG. 13, comprising FIG. 13A and FIG. 13B, depicts representative raft phase behavior in native membranes on the SLIM platform. FIG. 13A depicts representative images of native suspended bilayers made from GPMVs stained with Fast DiO (green, left), Cholera Toxin subunit B (red, middle), and the two merged (right). Scale bar=5 μm. FIG. 13B depicts the fluorescence trace of the line profile from FIG. 13A (white line) through two distinct bilayers that are enriched and not enriched in raft domains.

FIG. 14, comprising FIG. 14A through FIG. 14C, depicts representative functionality of a native suspended membrane. FIG. 14A depicts representative images of a reconstituted vesicle containing VAMP2 labeled with ATTO647-DOPE spontaneously fusing with a native bilayer. FIG. 14B depicts representative images of a reconstituted vesicle containing VAMP2 and SYT1, treated with CPX1, clamp on the surface and fuse with the native bilayer upon addition of calcium. FIG. 14C depicts a representative fluorescence trace revealing the moment the vesicle clamps to and fuses with the membrane upon addition of 1 mM Ca²⁺.

FIG. 15 depicts a schematic representation of vesicle fusion, representative images of vesicle fusion, and a representative fusion trace. Vesicles containing VAMP2 and SYT1 labeled with ATTO647-DOPE were added to a reconstituted bilayer containing pre-assembled t-SNAREs (SNAP25 and Syntaxin 1a). Vesicle fusion was recorded at 89 frames per second (11 ms/frame). Upon addition of calcium, the vesicle undergoes fusion as depicted in the representative images and the fluorescence trace. Inset: Fluorescence trace over a larger time scale.

FIG. 16 depicts a schematic representation of a method for using a chip device and applications for use.

### DETAILED DESCRIPTION

The present invention provides devices for examining native plasma membranes in a planar manner and methods of using the same. The devices are suitable for examining cell membranes, including the study of protein and protein-lipid complexes, lipid phase behavior, protein-protein interactions, membrane dynamics, ligand interactions, ion channels, protein trafficking, cell signaling, lipid and protein directionality, viral and bacterial interactions with the membrane, and drug screening. The devices support a variety of microscopies and spectroscopies for monitoring membranes, and their components, with single-molecule precision. The devices and methods also support physiologically relevant measurements including membrane potential, pH, compound influx and efflux, membrane stability, and membrane compositions.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used herein the term “cell surface molecule” refers to a peptide, polypeptide, binding domain, ligand, lipid, or carbohydrate that is directed to the extracellular surface of the host cell. The cell surface molecule may be anchored to the cell surface by covalent binding or non-covalent binding. The cell surface molecule may include a phospholipid, carbohydrate, or protein through which it attaches to the surface of the host cell. The cell surface molecule may be a polypeptide that binds to, or is conjugated to, a phospholipid, carbohydrate, or a polypeptide on the surface of the cell. For example, the polypeptide may use a phosphatidyl-inositol-glycan (GPI) anchor to attach to the surface of the cell, such as α-agglutinins, α-agglutinins, and flocculins. The cell surface molecule may also be a transmembrane protein.

As used herein, an “immunoassay” refers to any binding assay that uses an antibody capable of binding specifically to a target molecule to detect and quantify the target molecule.

As used herein the term “display molecule” refers to a molecule that can be localized to the surface of a target cell. The display molecule will typically comprise a first amino acid sequence to be displayed (e.g., a protein of interest, etc.) and a second amino acid sequence that anchors the display molecule to the surface of the target cell (e.g., a transmembrane domain, etc.). In certain instances the first and second amino acid sequences are linked in a single polypeptide. In an alternative embodiment, the first and second amino acid sequences may interact with each other to anchor the first amino acid sequence to the surface of a target cell. A display molecule may comprise a peptide, polypeptide, binding domain, ligand, lipid, or carbohydrate or combination thereof. The display molecule may also comprise a tag or peptide that can be labeled so as to detect binding of the display molecule to the cell surface, or sort cells displaying said molecule.

As used herein, components that are “fluidly connected” are connected to one another by a pipe, tube, line, channel, pore, or other equivalent means through which a fluid may flow from the first component to the second component or from the second component to the first component. Components that are fluidly connected need not be physically connected.

“Instructional material,” as that term is used herein, includes a publication, a recording, a diagram, or any other medium of expression which can be used to communicate the usefulness of the nucleic acid, peptide, polypeptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

“Measuring” or “measurement,” or alternatively “detecting” or “detection,” means assessing the presence, absence, quantity or amount (which can be an effective amount) of a given substance.

The term “reporter molecule” herein refers to a detectable compound or composition that can be detected by microscopy, spectroscopy, or other conventional techniques. The reporter molecule may be detected irrespective of environment or may undergo a physical or chemical change in certain environments which alters its detection. Reporter molecules may be macromolecules or small molecules. Examples of reporter molecules include, but are not limited to, isotopically labeled compounds, radioactive compounds, fluorescent compounds, phosphorescent compounds, optical dyes, and contrast dyes.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Membrane on a Chip

The present invention provides devices for supporting unilamellar lipid bilayers in an approximately native environment while maintaining native molecular orientations and methods of use thereof. The devices support the ability of proteins to diffuse freely in the reconstituted system. The devices are amenable to a variety of experimental setups and can be tuned to meet specific requirements.

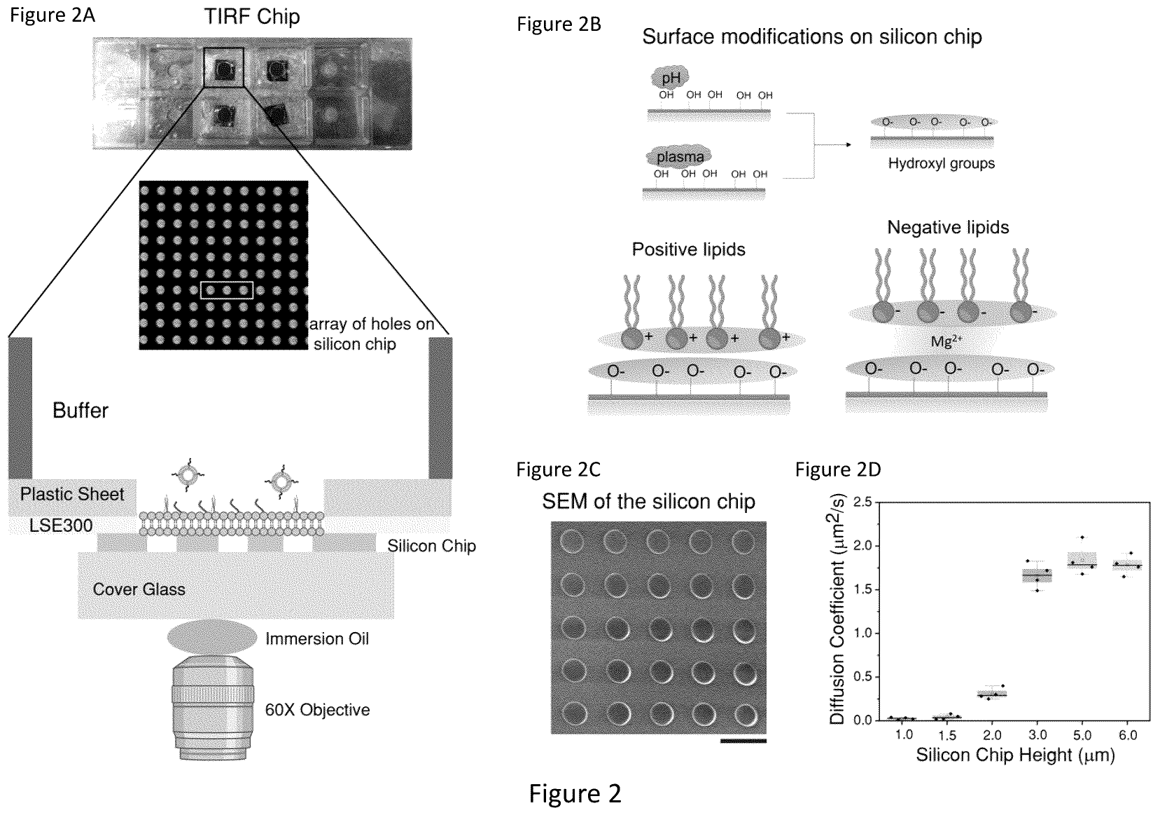

Referring now to FIG. 2A, an exemplary layout of membrane on a chip device is depicted. In one embodiment, the membrane on a chip device comprises a bottom layer of an essentially impermeable substrate having a first thickness; a chip layer of a second essentially impermeable substrate having a second thickness stacked on top of the bottom layer; and a barrier layer of a third essentially impermeable material having a third thickness stacked on top of the chip layer.

In various embodiments, the chip layer comprises a silicon chip. In some embodiments, the silicon chip comprises a plurality of holes, having a first diameter, which extend through the thickness of the chip. In some embodiments, the holes are arranged in an organized pattern. In some embodiments, the pattern consists of a series of rows and columns. In some embodiments, the rows and columns are arranged with a regular distance separating the holes of each row and each column. In some embodiments, the chip layer is stacked on top of the bottom layer such that they form a water-tight seal, whereby each hole in the chip layer is capable of containing a fluid. In some embodiments, the barrier layer is stacked on top of the chip layer such that they form a water-tight seal. In various embodiments, the barrier layer comprises one or more macro-holes, having a second diameter, which extend through the thickness of the barrier layer such that the holes of the chip layer are exposed, wherein fluid added to the macro-holes will flow freely into the holes if a membrane is not covering the holes.

In one embodiment, the membrane on a chip device further comprises a reservoir layer, with a fourth thickness, stacked on top of the barrier layer. In one embodiment, the reservoir layer is stacked on top of the barrier layer such that they form a water-tight seal, wherein macro-hole is exposed and fluid added to the reservoir layer will flow freely into the holes and macro-hole.

In some embodiments, the silicon chip comprises Si/SiO₂, silicon-borosilicate glass, and polydimethylsiloxane (PDMS). In one embodiment, the silicon chip comprises Si/SiO₂. In some embodiments, the silicon chip comprises a dopant. In some embodiments, the dopant is selected from the group consisting of phosphorus, arsenic, antimony, boron, aluminum, and gallium. In some embodiments, the dopant is phosphorus or aluminum.

In some embodiments, the silicon chip has a thickness of about 2.5 μm to about 6 μm. In one embodiment, the thickness is about 3 μm to about 6 μm. In one embodiment, the thickness is about 5 μm. In one embodiment, the thickness is 4.7 μm.

In some embodiments, the holes in the silicon chip are approximately cylindrical in shape. In some embodiments, the holes have a diameter of about 1 μm to about 10 μm. In one embodiment, the holes have a diameter of about 5 μm.

In various embodiments, the holes in the silicon chip are frustra. In some embodiments, the frustra are circular frustra. In some embodiments, the circular frustra are right circular frustra. In some embodiments, the frustra have an upper diameter at the top surface of the chip and a lower diameter at the bottom surface of the chip. In some embodiments, the lower diameter of the frustra is greater than the upper diameter. In some embodiments, the upper diameter is about 1 μm to about 10 μm. In some embodiments, the lower diameter is about 1.1 μm to about 15.4 μm.

In some embodiments, the plurality of holes in the silicon chip are disorganized or organized in a pattern. In some embodiments, the pattern comprises a series or rows and columns. In some embodiments, the rows and columns are perpendicular to each other. In one embodiment, the distance between the rows is equal to the distance between the columns. In one embodiment, the distance between rows and columns is approximately one half the diameter of the holes to approximately the diameter of the holes. In one embodiment, the array of holes comprises a series of perpendicular rows and columns wherein the distance between the rows is 5 μm and the distance between the columns is 5 μm. In some embodiments, the chip comprises a 200×200 array of holes.

In some embodiments, one or more of the holes in the silicon chip are fluidly connected. In one embodiment, the holes forming a row in the silicon chip are fluidly connected. In one embodiment, the holes forming a column in the silicon chip are fluidly connected. In one embodiment, a fluidly connected row of holes is fluidly connected, at the first end of the row, to a fluid input, through which a fluid may be introduced such that it flows through the fluidly connected holes, filling each with fluid, thereby granting access to both faces of the membrane. In one embodiment, the fluidly connected row of holes further is connected, at the second end of the row, to a fluid outlet, such that fluid may be introduced through the fluid inlet, flow through the fluidly connected row of holes, and out the outlet.

In some embodiments, each hole in the silicon chip holds between about 1 fL and about 1 nL. In some embodiments, each hole holds about 1 fL, about 2 fL, about 3 fL, about 4 fL, about 5 fL, about 6 fL, about 7 fL, about 8 fL, about 9 fL, about 10 fL, about 15 fL, about 20 fL, about 25 fL, about 30 fL, about 35 fL, about 40 fL, about 45 fL, about 50 fL, about 60 fL, about 70 fL, about 80 fL, about 90 fL, about 100 fL, about 110 fL, about 120 fL, about 130 fL, about 140 fL, about 150 fL, about 175 fL, about 200 fL, about 225 fL, about 250 fL, about 300 fL, about 350 fL, about 400 fL, about 450 fL, about 500 fL, about 600 fL, about 700 fL, about 800 fL, about 900 fL, about 1 pL, about 2 pL, about 3 pL, about 4 pL, about 5 pL, about 6 pL, about 7 pL, about 8 pL, about 9 pL, about 10 pL, about 15 pL, about 20 pL, about 25 pL, about 30 pL, about 35 pL, about 40 pL, about 45 pL, about 50 pL, about 60 pL, about 70 pL, about 80 pL, about 90 pL, about 100 pL, about 110 pL, about 120 pL, about 130 pL, about 140 pL, about 150 pL, about 175 pL, about 200 pL, about 225 pL, about 250 pL, about 300 pL, about 350 pL, about 400 pL, about 450 pL, about 500 pL, about 600 pL, about 700 pL, about 800 pL, about 900 pL, or about 1 nL.

In some embodiments, the bottom layer comprises a glass selected from the group consisting of fused silicate glass, soda-lime glass, borosilicate glass, SF11 glass, aluminosilicate glass, and glass-ceramic. In one embodiment, the glass is borosilicate glass.

In some embodiments, the bottom layer has a thickness about 20 μm to about 1 mm. In some embodiments, the bottom layer has a thickness of about 100 μm to about 1 mm. In one embodiment, the bottom layer has a thickness of about 150 μm.

In some embodiments, the bottom layer is attached to the silicon chip by anodic bonding, fusion bonding, or an adhesive. In one embodiment, the bottom layer is attached to the silicon chip by anodic bonding.

In some embodiments, the holes in the silicon chip comprise an electrode. In some embodiments, the electrode passes through the thickness of the bottom layer. In some embodiments, the electrode is flush with the top surface of the bottom layer, such that it is capable of making contact with fluid added to the hole but does not extend into the hole.

In some embodiments, the electrode comprises (poly(3,4-ethylenedioxythiophene)polystyrene sulfonate (PEDOT:PSS), silver, platinum, gold, copper, indium titanium oxide (ITO), and combinations thereof.

In some embodiments, the barrier layer comprises fused silicate glass, soda-lime glass, borosilicate glass, aluminosilicate glass, glass-ceramic, or plastic. In one embodiment, the barrier layer comprises plastic. Examples of suitable plastics include, but are not limited to, low-density polyethylene (LDPE), high-density polyethylene (HDPE), polyvinyl chloride (PVC), polystyrene (PS), polyamides (PA), epoxy resins, polypropylene (PP), polyesters, polyphenylsulfone, polyether ether ketone (PEEK), polyether block amides (PEBA), polymethylmethacrylate (PMMA), and polydimethylsiloxane (PDMS).

In various embodiments, the barrier layer is attached to the chip by anodic bonding, fusion bonding, or an adhesive. Examples of adhesives include, but are not limited to, epoxies, silicones, epoxy-polyurethane blends, acrylates, cyanoacrylates, and methylmethacrylates.

In some embodiments, the device comprises a reservoir layer. In some embodiments, the reservoir layer comprises silicate glass, soda-lime glass, borosilicate glass, aluminosilicate glass, glass-ceramic, or plastic. In one embodiment, the barrier layer comprises plastic. Examples of suitable plastics include, but are not limited to, low-density polyethylene (LDPE), high-density polyethylene (HDPE), polyvinyl chloride (PVC), polystyrene (PS), polyamides (PA), epoxy resins, polypropylene (PP), polyesters, polyphenylsulfone, polyether ether ketone (PEEK), polyether block amides (PEBA), polymethylmethacrylate (PMMA), and polydimethylsiloxane (PDMS).

In various embodiments, the reservoir layer is attached to the chip by anodic bonding, fusion bonding, or an adhesive. Examples of adhesives include, but are not limited to, epoxies, silicones, epoxy-polyurethane blends, acrylates, cyanoacrylates, and methylmethacrylates.

The present disclosure also provides unilamellar lipid bilayers, supported by a chip device of the present invention, in an approximately native environment while maintaining native molecular orientations. In one embodiment, the membrane on a chip comprises a device of the present disclosure in which a unilamellar lipid bilayer is suspended across the holes of the silicon chip and physically supported by the silicon chip. In some embodiments, the lipid bilayer is introduced by lysis of a vesicle or liposome. In some embodiments, the vesicle is an artificially generated vesicle. In some embodiments, the vesicle is derived from a cell. In some embodiments, the cell-derived vesicle is a giant plasma membrane vesicle (GPMV), small unilamellar vesicles (SUVs), large unilamellar vesicles (LUVs), and giant unilamellar vesicles (GUVs). Examples of cells from which a GPMV may be derived include, but are not limited to, N2a, HEK293, and INS-1. In some embodiments, the bilayer remains stable for about 1 hour, 2 hours, 3 hours, 4 hours, 5 hours, and 6 hours.

In a variety of embodiments, the holes of the silicon chip contain a fluid medium. In some embodiments, the fluid medium comprises a fluid selected from the group consisting of water, aqueous salt solutions, aqueous buffers, cell culture media, index matching media, and combinations thereof. In one embodiment, the fluid media comprises an aqueous buffer and index matching media. In one embodiment, the index matching media comprises OptiPrep™ (iodixanol). In one embodiment, the index matching medium is Nycodenz® (iohexol). In some embodiments, the matching media contains a combination of OptiPrep™ and sucrose. In some embodiments, the matching medium has a refractive index greater than about 1.4. In some embodiments, the matching medium has a refractive index between about 1.4 and about 2. In some embodiments, the matching medium has an osmolarity of approximately 1300 mOsm, 1400 mOsm, 1500 mOsm, and 1600 mOsm. In some embodiments, the matching medium has an osmolarity of approximately 1462 mOsm. In some embodiments, the matching medium has an osmolarity about 1300 mOsm, about 1200 mOsm, about 1100, about 1000 mOsm, about 900 mOsm, about 800 mOsm, about 700 mOsm, 1 about 600 mOsm, about 500 mOsm, about 400 mOsm, about 300 mOsm, about 200 mOsm, about 150 mOsm, about 140 mOsm, about 130 mOsm, or about 128 mOsm.

### Systems

The present invention also provides systems for examining a membrane on a chip device of the present invention. In various embodiments, a system of the invention comprises a chip device of the present invention and a microscope, spectrometer, spectrophotometer, plate reader, pH meter, any other analog or digital measuring device, and/or an analog or digital device for readout.

In some embodiments, the system comprises a membrane on a chip device of the present invention and a microscope. In some embodiments, the microscope is an optical or electron microscope. In one embodiment, the optical microscope is an optical microscope equipped for TIRF microscopy.

In some embodiments, the system comprises a membrane on a chip device of the present invention and one or more spectrometers and/or spectrophotometers. In some embodiments, the one or more spectrometers and/or spectrophotometers are one or more selected from the group consisting of a Raman spectrometer, infrared spectrometer, UV/Vis spectrophotometer, a mass spectrometer, and a nuclear magnetic resonance spectrometer.

### Methods

The present invention also provides method of using the disclosed devices. Methods of using the device can involve any number of different experiments, including, but not limited to, examining cell membranes, including the study of protein and protein-lipid complexes, lipid phase behavior, protein-protein interactions, membrane dynamics, ligand interactions, ion channels, protein trafficking, cell signaling, lipid and protein directionality, viral and bacterial interactions with the membrane, and drug screening. Methods of using the device also include experiments involving physiologically relevant measurements including membrane potential, pH, compound influx and efflux, membrane stability, and membrane compositions. These methods may involve or include any number of microscopy, spectroscopy, electrochemical, electrophysical, electro-optical, and/or electroacoustic techniques or experiments.

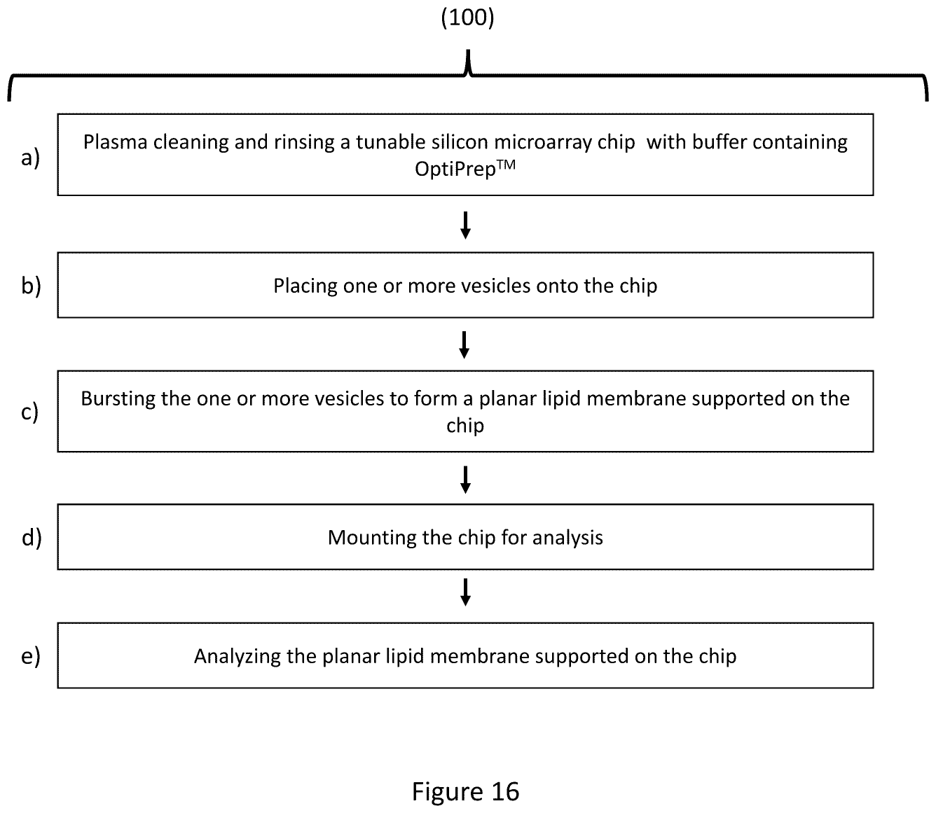

Referring now to FIG. 16, shown is a diagram depicting an exemplary method 100 for utilizing a tunable silicon microarray chip according to aspects of the present invention. In some embodiments, method 100 comprises the steps of: a) plasma cleaning and rinsing a tunable silicon microarray chip; b) placing one or more vesicles on the chip; c) bursting the vesicles to form a planar lipid membrane supported on the chip; d) mounting the chip for analysis; and e) analyzing the planar lipid membrane supported on the chip.

In some embodiments, step a) comprises rinsing the chip device with a buffer. Examples of suitable wash buffers include, but are not limited to, sterile water, saline, phosphate buffered saline (PBS), tris(hydroxymethyl)aminomethane (Tris), 4-(2-hydroxyethyl)-1-piperazineethanesulfonic acid (HEPES), and piperazine-N,N′-bis(2-ethanesulfonic acid (PIPES). Other additional components of the wash buffers that may be useful include salts, reducing agents, and detergents. In some embodiments, the buffer is supplemented with 20% OptiPrep™, 25% OptiPrep™, 35% OptiPrep™, 40% OptiPrep™, 45% OptiPrep™, 50% OptiPrep™, 55% OptiPrep™, 60% OptiPrep™, 65% OptiPrep™, and 70% OptiPrep™. In some embodiments, the buffer is supplemented with 1 mM MgCl₂, 2 mM MgCl₂, 3 mM MgCl₂, 4 mM MgCl₂, 5 mM MgCl₂, 6 mM MgCl₂, 7 mM MgCl₂, 8 mM MgCl₂, 9 mM MgCl₂, and 10 mM MgCl₂.

In some embodiments, step b) comprises incubating the vesicles for 5 minutes, 10 minutes, 15 minutes, 20 minutes, 25 minutes, and 30 minutes. In some embodiments, step b) comprises incubating the chip in a solution containing vesicles.

In some embodiments, the vesicles added to the chip in step b) are cell-derived vesicles. In some embodiments, the vesicles are reconstituted vesicles.

In some embodiments, step c) comprises rupturing vesicles by centrifugation. In some embodiments, the vesicles are centrifuged at about 50×g, 100×g, about 150×g, about 200×g, or about 300×g. In some embodiments, step c) comprises rupturing vesicles by osmotic shock. [[Inventors, please provide any additional details that you believe are important for rupturing vesicles on the chip.]]

In some embodiments, step e) comprises performing microscopy or spectroscopy experiments the lipid membrane supported by a device of the present invention. Methods comprising microscopy or spectroscopy experiments can be used for monitoring membranes, their components, and their interactions with single-molecule precision. Examples of microscopy techniques that may be employed include, but are not limited to, bright field, oblique illumination, dark field, phase contrast, differential interference contrast (DIC), internal reflection, total internal reflection (TIR), total internal reflection fluorescence (TIRF), fluorescence, confocal, two-photon, light sheet fluorescence, wide-field multiphoton super-resolution, ultraviolet, infrared, digital holographic, laser, laser scanning, photoacoustic, photoactivated localization, spinning disk confocal, structure illumination, selective plane illumination, stimulated emission depletion, stochastic optical reconstruction, variable-angle epifluorescence, and electron microscopy. Examples of spectroscopy techniques that may be employed include, but are not limited to, infrared, Raman, small-angle x-ray scattering, and ultraviolet-visible spectroscopy.

The microscopy and spectroscopy techniques may be employed as stand-alone techniques or may be coupled with other techniques. Techniques which may be coupled to the microscopy or spectroscopy techniques include, but are not limited to, Forster resonance energy transfer (FRET), acceptor bleaching, sensitized emission, polarization anisotropy, fluorescence lifetime imaging microscopy (FLIM), fluorescence recovery after photobleaching (FRAP), fluorescence loss in photobleaching (FLIP), fluorescence localization after photobleaching (FLAP), Dexter electron transfer, surface energy transfer, time-resolved fluorescence energy transfer, bimolecular fluorescence complementation (BiFC), luciferase complementation, and split-ubiquitin.

In some embodiments, where the device comprises electrodes, the methods may comprise measurement of charge gradient, charge flow, proton gradient, proton flow, gradient of an ion of interest, flow of an ion of interest across a membrane, and capacitance.

The following are given purely as exemplary methods, which should not be construed as limiting of the scope of the invention.

In one embodiment, a method of the present invention comprises examining interactions between a protein and a protein, lipid, nucleic acid, glycoside, or other small molecule comprising tagging the protein(s), lipid, nucleic acid, glycoside, or other small molecule with one or more fluorescent labels and observing the interactions by a microscopy technique such as TIRF microscopy, which may further be combined with a technique such as FRAP. In another embodiment, a method of the present invention comprises examining interactions between a protein and a protein, lipid, nucleic acid, glycoside, or other small molecule comprising tagging the protein with a FRET donor and the other protein, lipid, nucleic acid, glycoside, or other small molecule with a FRET acceptor, and observing the interactions by a microscopy technique such as TIRF microscopy. In another embodiment, the protein may be tagged with a FRET acceptor and the other protein, lipid, nucleic acid, glycoside, or other small molecule tagged with a FRET donor.

In one embodiment, a method of the present invention comprises examining ion channels comprising preparing a lipid membrane containing the ion channel(s) of interest across the holes of the chip, adding a molecule of interest which may or may not interact with the ion channel, and detecting a change. In one embodiment, the change is a change in a reporter molecule added to the holes of the chip prior to preparation of the lipid membrane and monitoring the reporter molecule by any conventional microscopy or spectroscopy technique. In another embodiment, the change may be detected by a change in membrane potential, pH, or membrane stability.

In one embodiment, a method of the present invention comprises screening drugs comprising preparing a device of the present invention in which a putative drug target of interest is embedded in the membrane spanning the holes of the chip, introducing the panel/library of drugs to be screened, and detecting a change. In one embodiment, the change is a change in a reporter molecule added to the holes of the chip prior to preparation of the lipid membrane and monitoring the reporter molecule by any conventional microscopy or spectroscopy technique. In another embodiment, the change is a change in the interaction of one or more proteins, lipids, nucleic acids, glycosides, and/or other small molecules. In another embodiment, the change may be detected by a change in membrane potential, pH, or membrane stability.

In one embodiment, a method of the present invention comprises nanopore sequencing of nucleic acids or polypeptides as they traverse a pore in the suspended membrane. In some embodiments, the nucleic acids are DNA, RNA, or a combination thereof.

### Kits

The present invention also pertains to kits useful in the methods of the invention. Such kits comprise various combinations of components useful in any of the methods described herein, including, for example, a chip of the present invention, and/or vesicles for generating a unilamellar plasma membrane on the chip, buffers and solutions including the index matching medium and/or a stabilization agent and/or instructional material.

In some embodiments, the kit comprises vesicles that are freeze-dried or suspended in a stabilization agent for long term storage and shipping. In some embodiments, the stabilization agent is a polymer. In some embodiments, the vesicles are artificially generated vesicles. In some embodiments, the vesicles are native vesicles obtained from cells.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples, therefore, specifically point out exemplary embodiments of the present invention, and are not to be construed as limiting in any way the remainder of the disclosure.

To address the challenges of designing a system for examining membranes in a native environment, it was determined that pore-spanning suspended bilayers are best suited. Their main advantages are the well-defined geometry to mimic the in vivo environment, the ability to reproduce biological processes at the membrane interface accurately, and provide long-term membrane stability suitable for time-lapse imaging (Hubrich, R., et al., 2019, Biophysical Journal, 116(2):308-318; Muhlenbrock, P., et al., 2020, Biophysical Journal, 119(1):151-161); Muhlenbrock, P., et al., 2021, European Biophysics, 50(2):239-252; Spindler, S., et al., 2018, Nano Letters, 18(8):5262-5271; Heo, P., et al., 2019, Small, 15(21): e1900725; Ramakrishnan, S, et al., 2018, Langmuir, 34(20):5849-5859; Bera, M., et al., 2022, eLife, 11:e71938; Coleman, J., et al., 2018, Cell Reports, 22(3): 820-831; Ramakrishnan, S., et al., 2019, FEBS Letters, 593(2): 154-162; Ramakrishnan, S., et al., 2020, eLife, 9:e54506). Total internal reflection fluorescence (TIRF) microscopy is the primary method of choice for selective imaging of single fluorescent molecules, studying molecular trafficking events at the plasma membrane, and spatial-temporal dynamics of intracellular biochemical events in living cells (Axelrod, D., et al., 2008, Methods in Cell Biology, 89:169-221). The integration of TIRF-based single-molecule imaging capabilities into a suspended membrane platform is critical to revolutionizing lipid bilayer investigations.

The suspended lipid membrane (SLIM) platform described herein allows the pore-spanning suspended bilayer to be integrated into the TIRF system. The system exploits the total internal reflection principle to achieve a high signal-to-noise ratio in a suspended bilayer environment. This platform can perform quantitative single-molecule imaging on suspended lipid bilayers using a generic total internal reflection microscope Cellular plasma membranes were utilized to recreate a system with conserved native protein and lipid asymmetries. Cells regulate lipid asymmetry and protein orientation through well-controlled mechanisms. Loss of asymmetry is a characteristic of cancer cells and has important implications in cancer detection and therapy (Ran, S., et al., 2002, International Journal of Radiation Oncology, Biology, Physics, 54(5): 1479-1484). Although most studies use symmetric bilayers to understand protein functions, little is known about the role of asymmetric membranes in regulating protein complexes. Current approaches to investigate cell membranes often involve depositing cells on a cover glass and unroofing them to eliminate cytoplasmic contents. This approach is as harsh as fixing the cells (i.e., freezing the lateral mobility of proteins similar to the supported bilayers) before permeabilizing them. A gentler approach was developed using giant plasma membrane vesicles (GPMVs) (>15 μm) harvested from cells to recreate the native cell plasma membrane on a chip in a planar geometry suitable for TIRF imaging. The directionality of membrane lipids and proteins is preserved, paving the way for recreating an asymmetric planar cell membrane on a chip.

Using the SLIM platform, visualization of vesicle fusion was achieved by the minimal fusion machinery such as SNAREs, synaptotagmin, and complexin at an ˜11 msec/frame time scale. The number of proteins under a docked vesicle were also able to be counted and fluorescent resonant energy transfer (FRET) visualized on this freestanding membrane system. The membranes were characterized using lipid and protein fluorescence recovery after photobleaching (FRAP), ion transport across the membranes, and molecule trapping. The combination of these tests proved the biological functionality of the platform. This significantly benefits applications like single-molecule imaging or super-resolution microscopy, colocalization, Förster resonance energy transfer (FRET), single-particle tracking, and lipid and protein dynamics.

### Example 1: The SLIM Platform: Dynamic Imaging of Individual Protein Macromolecules

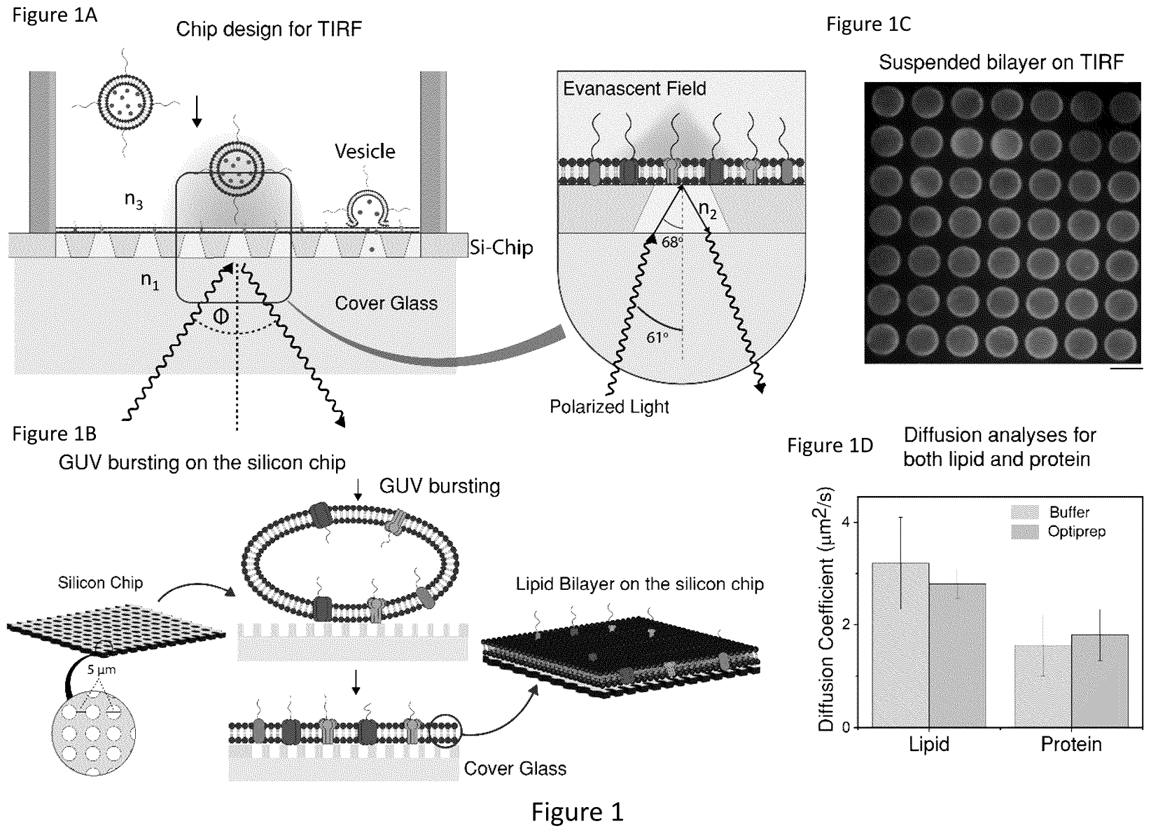

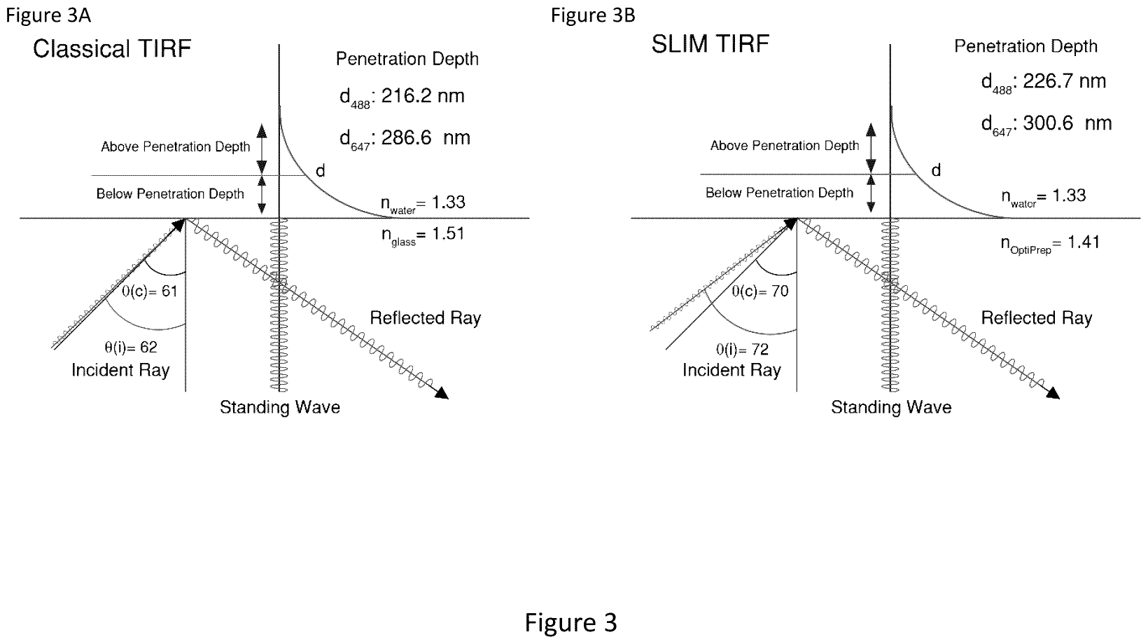

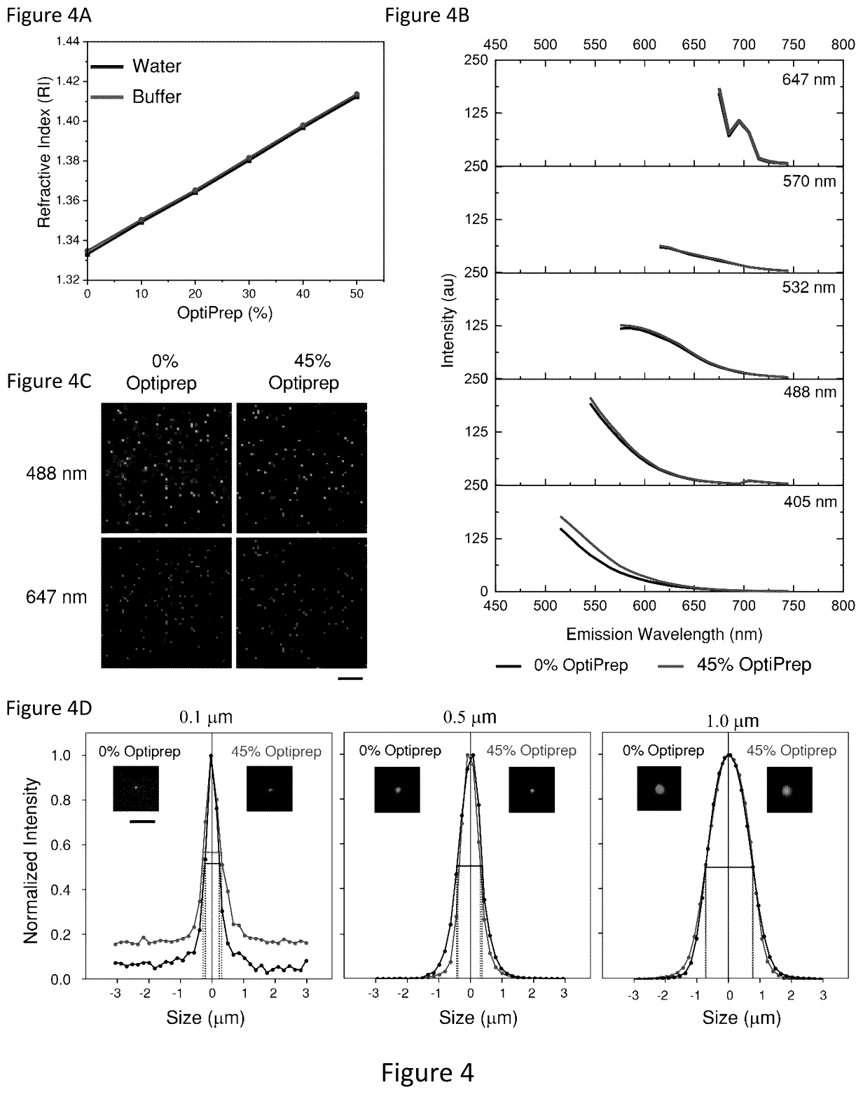

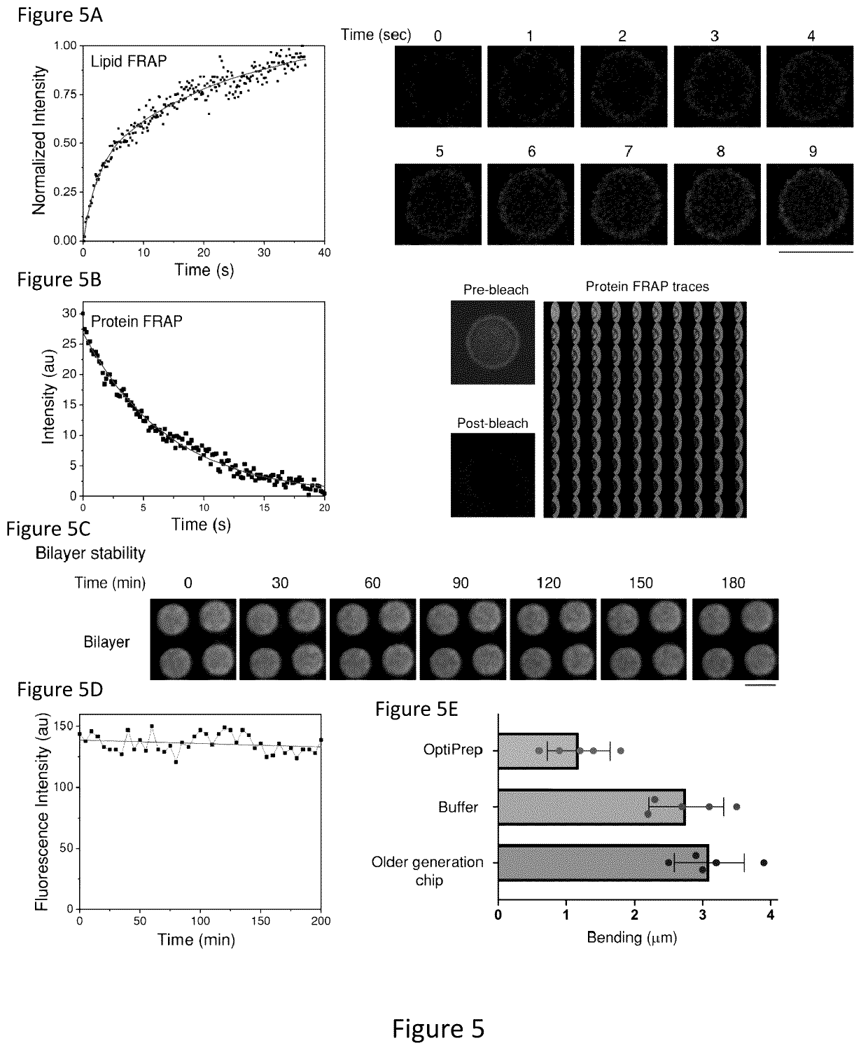

In order to examine membrane dynamics in a native setup, a system was developed to detect real-time dynamic single molecules on a suspended lipid bilayer (FIG. 1). A thin tunable silicon microarray chip (5 mm length×5 mm width×4.7 μm depth) was designed to contain an array of 200×200 cylindrical holes with a diameter of 5 μm, which was mounted onto a 150 μm thick glass coverslip to prevent chip damage as well as to match the objective's refractive index (n=1.5) (FIGS. 2A and 2C). Each well in the array can hold up to ˜ 92 fL of solution. The fabricated Si chips were cleaned, checked for flatness using a dial gauge, and checked for leaks before use. Prior attempts at platform design helped determine that a minimum chip height requirement of 3 μm above the glass coverslip was required to prevent pore-spanning bilayers from coming into contact with the glass support (FIG. 2D) A major challenge addressed in integrating the pore-spanning system for TIRF microscopy was getting the incident light from the oil-glass interface to the bilayer (4.7 μm above the glass surface). This was solved using an intermediate index-matching medium (n₂) between the glass (n₁) and bilayer (n₃). It was calculated that an n₂=1.41 was required to achieve TIRF at the bilayer interface (FIG. 3B). To arrive at the appropriate medium, sucrose and Iodixanol (OptiPrep™) were used to create a refractive index greater than 1.4. Sucrose (RI_{1.4} required a 50% (w/v) solution with an osmolarity of ˜1462 mOsm), created a huge osmolarity difference between the index-matching medium and the physiological buffer, resulting in the rupturing of suspended lipid membranes. Iodinated density gradient media such as OptiPrep™ (RI_{1.4} required a 45% (w/v) solution with an osmolarity of 128 mOsm) cause significantly smaller osmolarity differences, therefore OptiPrep™ was chosen for the index matching media (n₂) (FIG. 4A).

Low autofluorescence is another essential requirement of a buffer to be used in single-molecule imaging applications. A spectral emission scan of 0% and 45% aqueous OptiPrep™ solutions showed no difference in immunofluorescence compared to the buffer at the commonly used excitation wavelengths (FIG. 4B). Next, to evaluate any optical aberrations of 45% OptiPrep™ on image quality, the point spread functions of 100 nm, 500 nm, and 1 μm fluorescent beads were quantified (FIGS. 4C and 4D), with no significant distortions to lateral and axial image resolution observed compared to controls in buffer without OptiPrep™ (RI=1.33, FIGS. 4C and 4D). Overall, the physicochemical properties of OptiPrep™ proved suitable for refractive index matching applications.

Initial tests on the platform were performed with reconstituted membranes. Using a well-established protocol, giant unilamellar vesicles (GUVs) were burst to form planar lipid membranes on the platform (Ramakrishnan, S., et al., 2018, Langmuir, 34(20): 5849-5859; Bera, M., et al., 2022, eLife, 11:e71938; Coleman, J., et al., 2018, Cell Reports, 22(3):820-831; Ramakrishnan, S., et al., 2019, FEBS Letters, 593(2): 154-162; Ramakrishnan, S., et al., 2020, eLife, 9:e54506). FIG. 1C shows TIRF images of pore spanning lipid bilayer spread on the p-doped silicon. The coverage of the membrane depends on the silicon doping, the nature of the lipids, and the hydrophilicity protocol used. The SLIM platform can be tailored to support positively or negatively charged lipid bilayers by tuning the silicon doping with either n-type or p-type silicon (FIG. 2B). Due to the opacity of silicon, only the bilayer spread on top of holes is visible. Next, tests were performed to validate the unilamellar bilayer.

### Example 2: Suspended Lipid Membranes Mimic In Vivo Environments

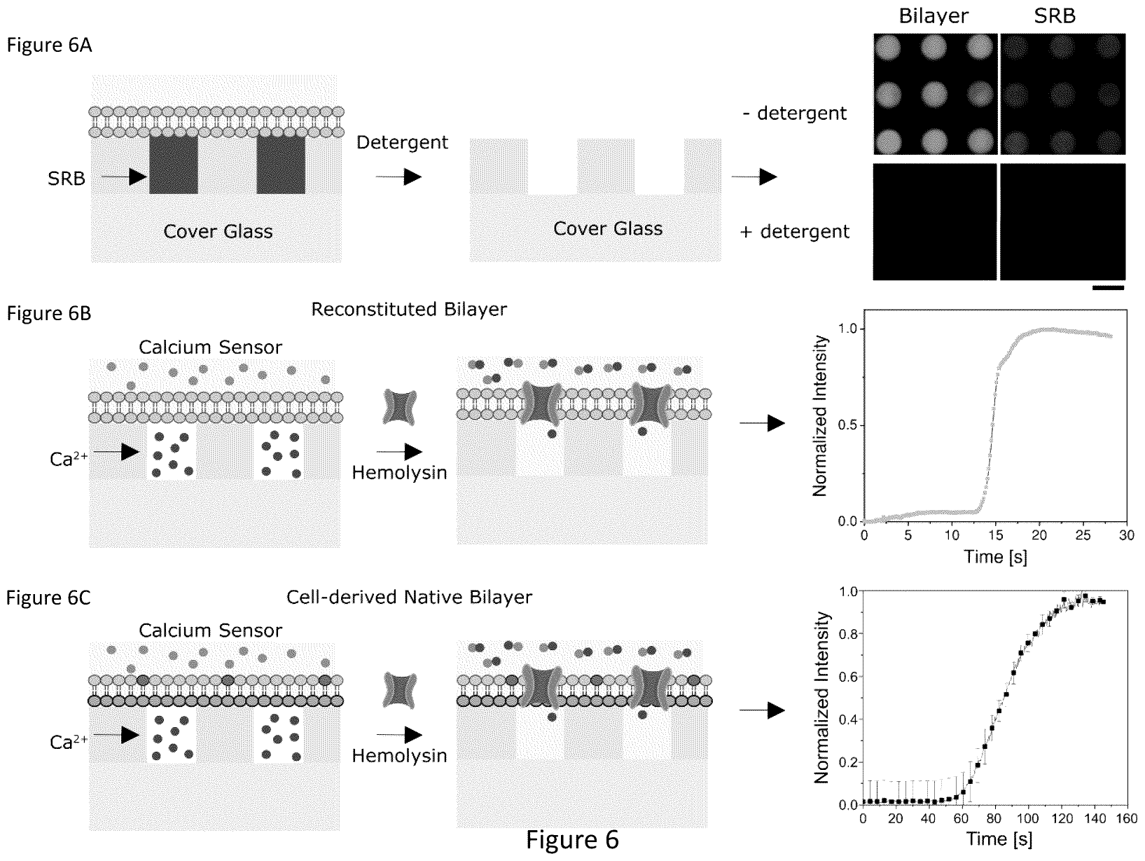

A quantitative analysis of lateral diffusion characteristics of lipids and proteins present on the SLIM platform was performed using fluorescence recovery after photobleaching (FRAP). In all cases, the recorded fluorescence intensity fully recovered (FIG. 5A), demonstrating that the movement of lipid molecules in both leaflets is mainly due to Brownian motion, migrating over the substrate and maintaining continuity across the holes. To verify that the OptiPrep™ index-matching medium does not affect the lipid diffusion in reconstituted bilayers, lipid diffusion was measured with and without 45% OptiPrep™. The obtained diffusion coefficients of 3.2±0.9 μm²/s and 2.8±0.3 μm²/s, respectively, suggest that OptiPrep™ does not significantly affect lipid diffusion (FIG. 1D). A homogeneous fluorescence distribution was observed across the hole, showing that the bilayers are continuous and freestanding over the holes, and the bilayer remains stable over 3 hours (FIG. 5C).

To verify the diffusivity of the transmembrane domain-containing proteins incorporated in the bilayers, Alexa 488-labeled t-SNAREs were reconstituted into the suspended bilayer. Proteins tend to diffuse within the holes in the pore-spanning membranes and cannot be exchanged between the hole and the substrate When proteins diffuse to the edge of the holes, they adhere to the silicon substrate. In this scenario, a classical FRAP approach cannot be employed to look for the recovery of protein molecules. An elliptical region of interest (ROI) covering 25-30% of the hole was used to monitor the bleaching of diffusing proteins over time (FIG. 5B). The ability of proteins to diffuse freely in the reconstituted system provides a cell-like native environment to exhibit their properties and interact with their partners. Protein diffusion coefficients of 1.6±0.6 μm²/s and 1.8±0.5 μm²/s were estimated from characteristic bleaching times in reconstituted membranes for buffer and OptiPrep™ conditions, respectively (FIG. 1D), further verifying that OptiPrep™ does not affect protein diffusion.

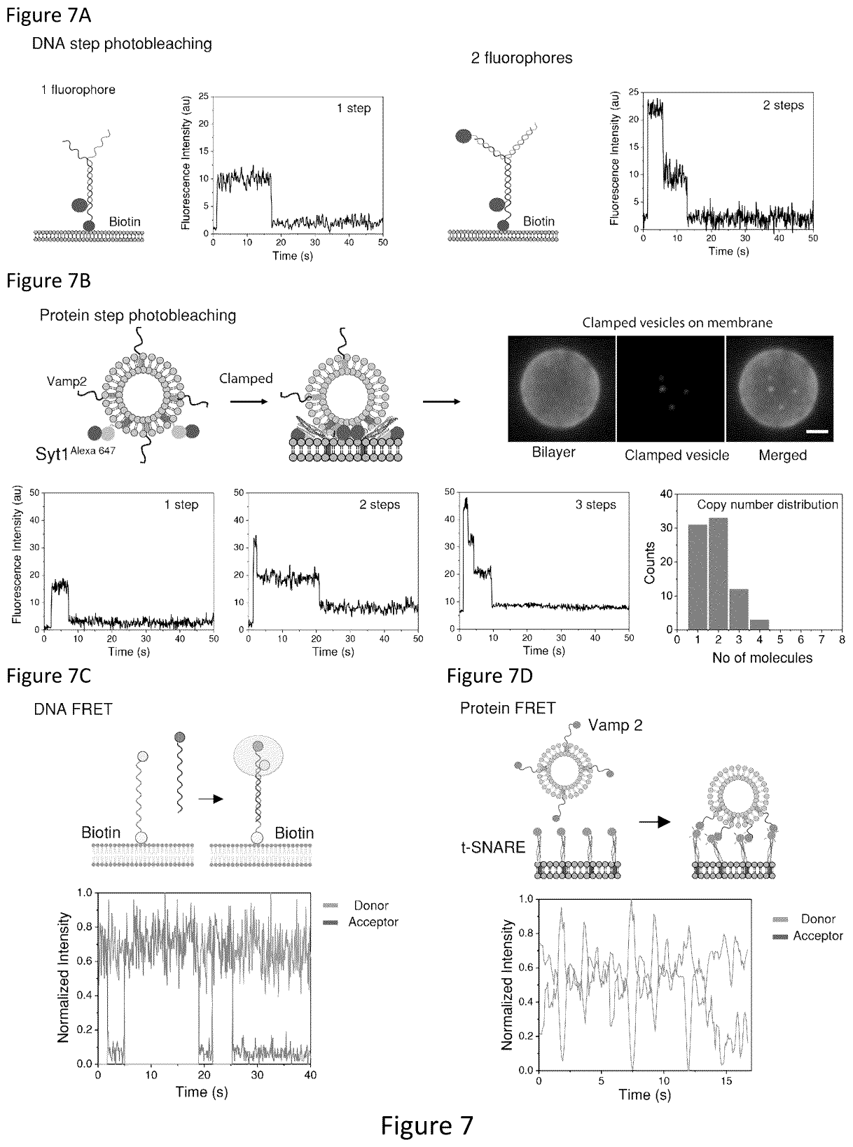

To verify that the lipid bilayers formed on the thin silicon chip have separate intra- and extracellular environments, soluble sulforhodamine (SRB) was trapped below the bilayer. A successive wash with a detergent solution solubilized the lipid membrane, releasing the captured fluorophore (FIG. 6A), confirming that the lipid bilayer formation on the chip creates separate aqueous compartments above and below the bilayer, mimicking intra- and extracellular compartments. Creating a planar unilamellar bilayer is equally essential to performing biological assays. To verify the existence of a single bilayer, α-hemolysin, which is known to integrate into and form a channel within a single bilayer due to the transmembrane domain depth of 5.2 nm, was used (Song, L., et al., 1996, Science, 274(5294): 1859-1865). α-hemolysin is a 33 kDa water-soluble monomer that becomes a heptamer when bound to phosphocholine lipids, forming a pore with a diameter of 1.4-2.4 nm (Song, L., et al., 1996, Science, 274(5294): 1859-1865). Calcium chloride was trapped below the bilayer and a cell-impermeable calcium-sensitive fluorophore was added above the bilayer. With the addition of α-hemolysin, a rapid fluorescence signal increase was observed, suggesting Ca²⁺ ions effluxed through the α-hemolysin pores, validating the unilamellar membrane behavior (FIG. 6B).

### Example 3: Single-Molecule Imaging on Suspended Lipid Membranes

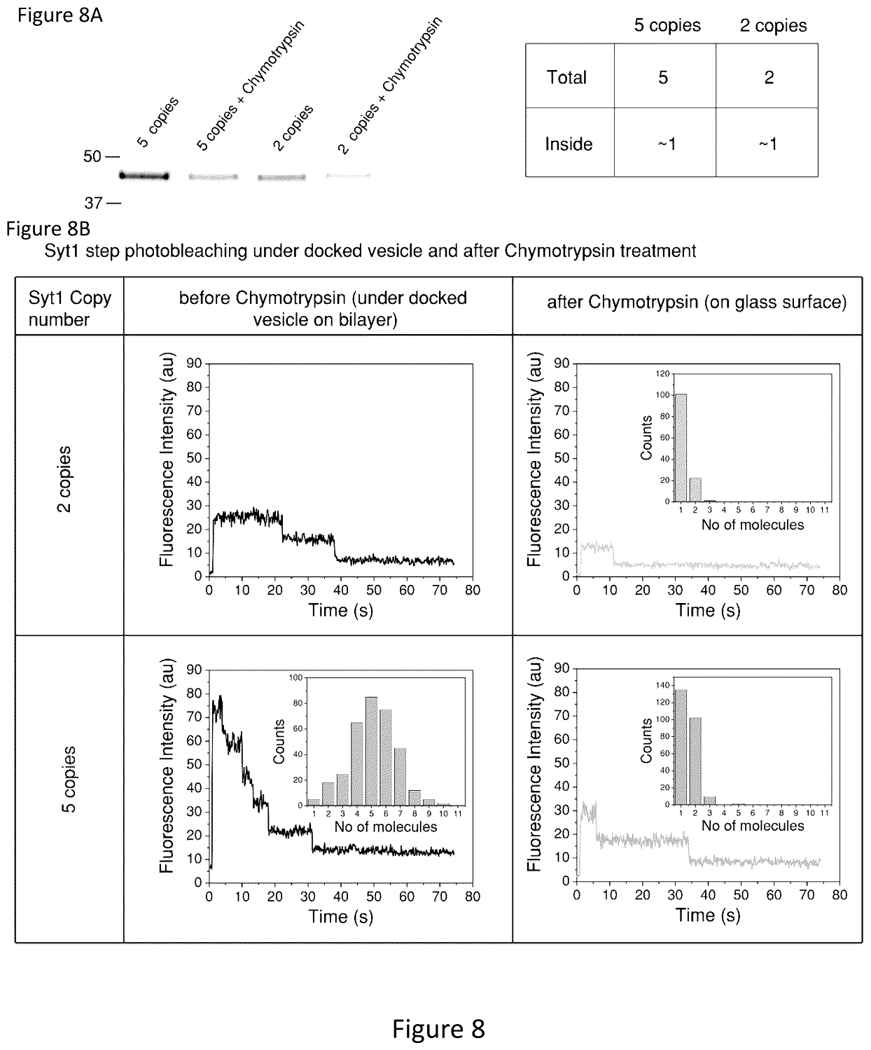

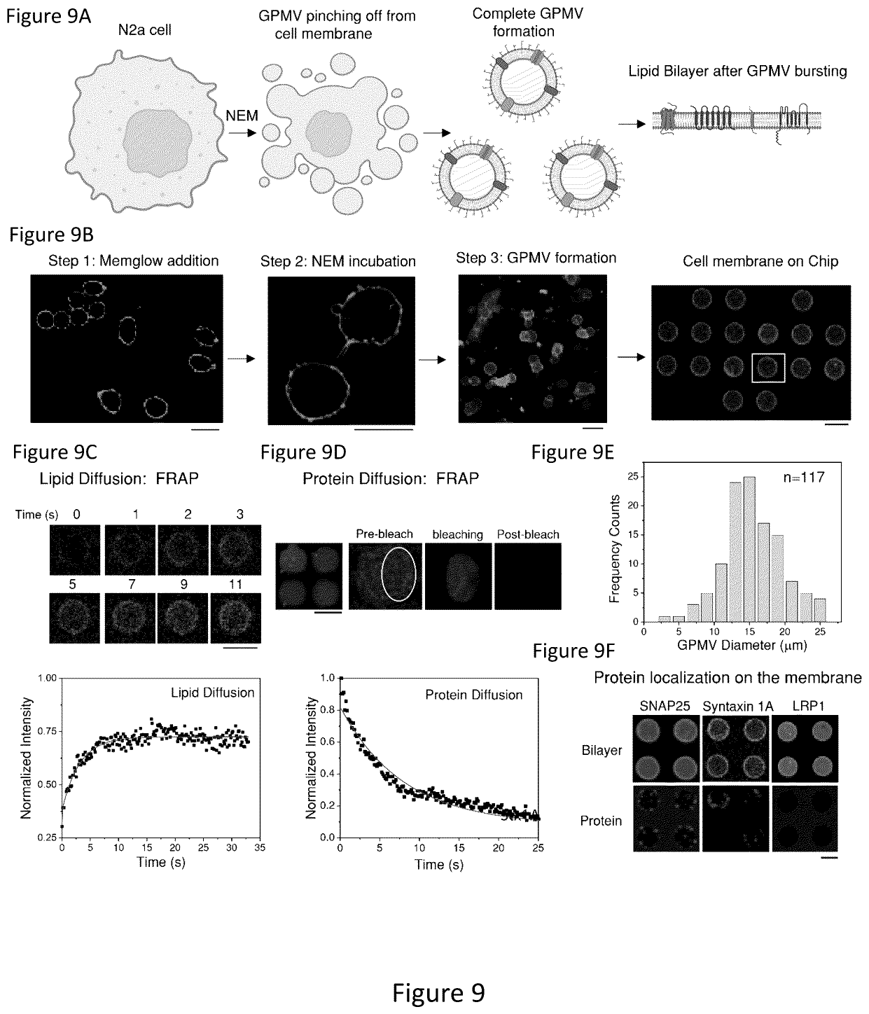

TIRF microscopy allows visualization of single-molecule biological interactions with high temporal resolution and high signal-to-noise ratio. To test the ability to detect single fluorescent molecules on the suspended lipid bilayer, Y-shaped DNA, composed of three overlapping DNA strands partially complementary to each other, was synthesized in a single step by simple mixing. The Y-shaped DNA, that contained one or two fluorophores, was attached to the lipid bilayer using streptavidin/biotin interactions (Chatterjee, S., et al., 2012, Nanoscale, 4(5):4568-1571). Stepwise photobleaching is the gold standard for determining the copy number of proteins and subunits in a fluorescent spot (Verdaasdonk, J. S., et al., 2014, Methods in Cell Biology, 123:347-365; Li, F., et al., 2021, FEBS Letters, 595(17):5185-2196). Based on the bleaching traces obtained, constructs containing one fluorophore vs. two fluorophores could be distinguished (FIG. 7A). This was further applied to vesicles containing VAMP2 and either 2 or 5 copies of fluorescently labeled Synaptotagmin-1 (Syt1) molecules. Syt1 protein molecules dock on PIP2-containing clusters on the membrane such that the Syt1 proteins facing outside of the vesicles are bound to PIP2 clusters and don't move due to strong interactions between polybasic patches of C2B domains within Synaptotagmin 1 and PIP2 lipids, thus contributing to discrete bleaching traces (Park, Y., et al., 2015, Nature Structural & Molecular Biology, 22(10): 815-823). When looking at the number of molecules under a docked vesicle, up to 4 steps could be discerned for vesicles containing 2 Syt1 molecules (FIG. 7B) and 7 steps for vesicles containing 5 copies (FIG. 8B), suggesting this modality allows molecular counting with single-molecule precision. These results suggest that there is a heterogeneity of the protein occupancy in reconstituted liposomes, in line with previous reports (Cliff, L., et al., 2020, Biochimica et Biophysica Acta—Biomembranes, 1862(1):183033). To further dissect the contribution of labeled molecules facing outward vs. inward in the liposome, the proteins facing the outside of the liposomes were digested using chymotrypsin, after which one or two-step bleaching traces were observed, indicating asymmetric reconstitution of Syt1 (FIG. 8B). Such reconstitution behavior is observed with proteins containing large cytoplasmic domains and liposomal lipid composition (Ramakrishnan, S., et al., 2019, FEBS Letters, 593(2). 154-162; Ramakrishnan, S., et al., 2020, eLife, 9:e54506; Marek, M., et al., 2011, Journal of Biological Chemistry, 286(24):21835-21843; Hickey, K. D., et al., 2011, Journal of Lipids, 2011:208457). The versatile strategy presented here is a powerful tool for counting proteins present in vesicles and can be extended to count a variety of membrane proteins reconstituted into vesicles by incorporating tags such as SNAP and HALO, which allow for quick and robust labeling.

Next, it was tested if molecular changes could be tracked with the benefit of single-molecule precision. DNA strands containing either Cy3 or Cy5 were used as donor and acceptor molecules, anchored to the bilayer via biotin/streptavidin interactions (McCann, J. J., et al., 2010, Biophysical Journal, 99(3):961-970) FRET was consistently observed between the strands (FIG. 7C). The setup was also tested to see if similar interactions could be detected between protein domains. Labeled VAMP2 and SNAP25 allowed visualization of the N-terminal zippering of the SNAREs (Krishnakumar, S. S., et al., 2011, Nature Structural and Molecular Biology, 18(8):934-940). Using Alexa Fluor 568 and Alexa Fluor 647 as donor and acceptor, respectively, FRET was able to be visualized after the addition of vesicles to the bilayers containing labeled SNAP25 (FIG. 7D). Different intensities of FRET could be visualized, suggesting different numbers of SNARE pins engaging. This demonstrates that the system can be used to monitor protein interactions using FRET.

### Example 4: Single-Molecule Imaging of Native Cell Membranes on a Chip

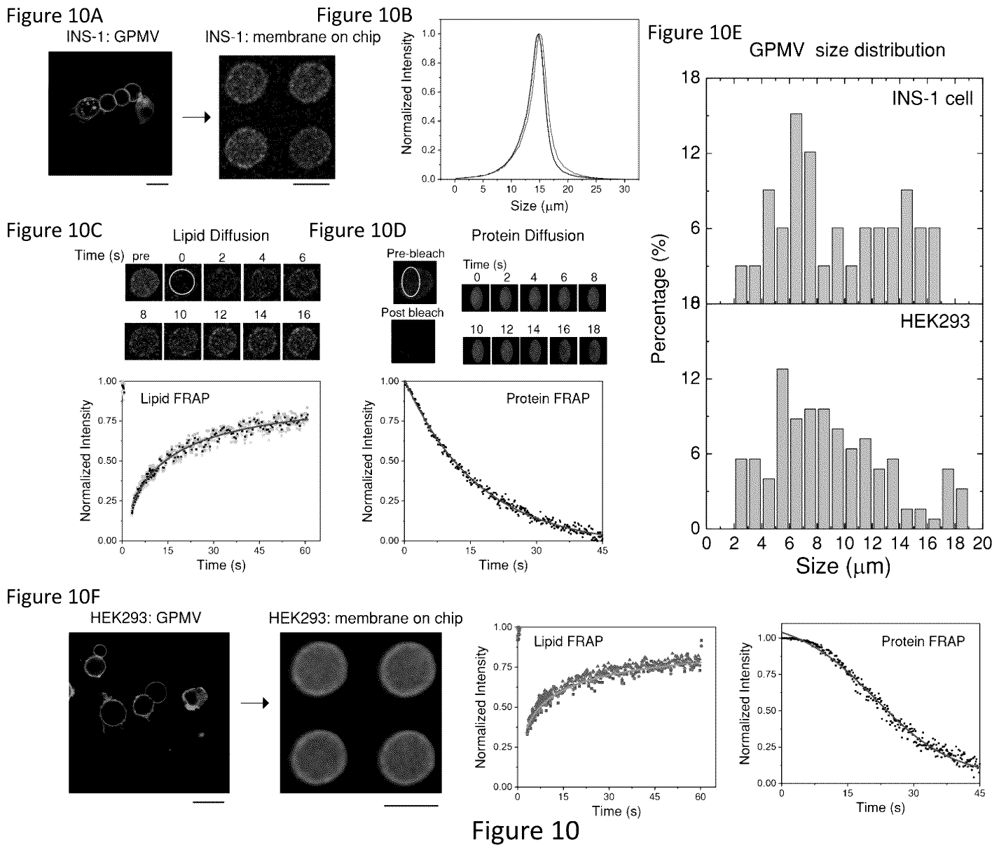

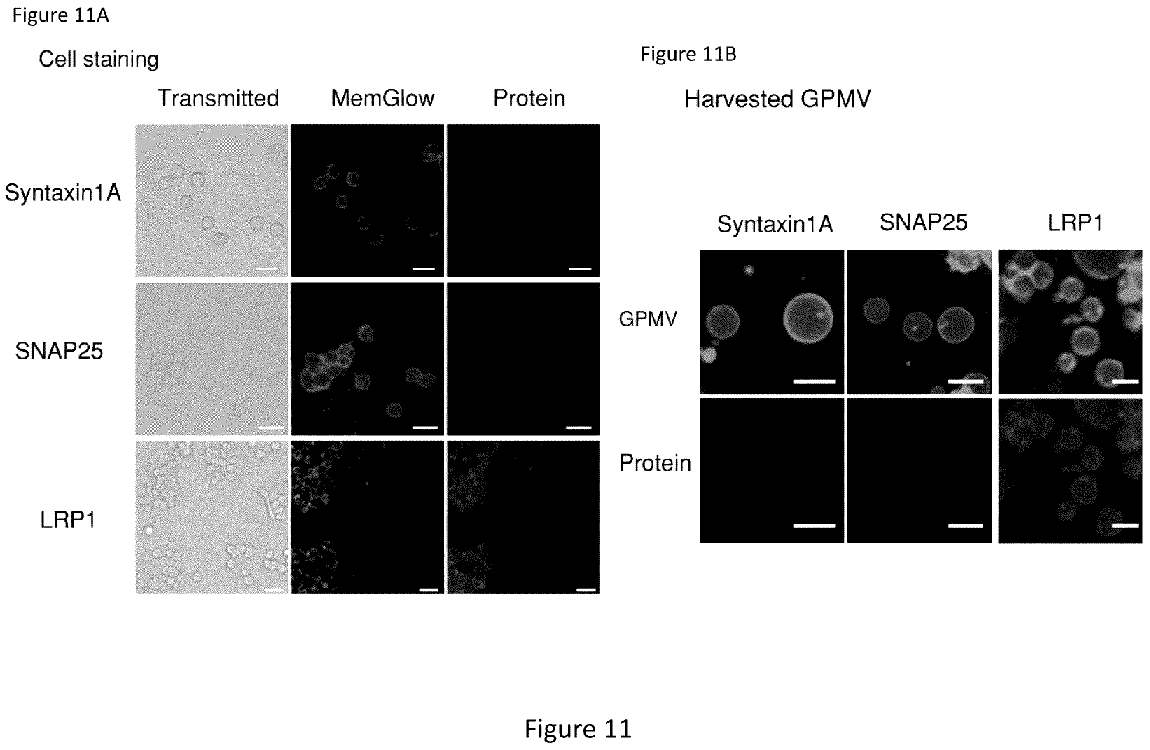

Reconstituted bilayers cannot capture the full complexity of the cellular plasma membrane. This limitation was overcome by harvesting giant plasma membrane vesicles (GPMVs) from cells. This method provides physiological native membranes in a controlled in vitro environment (Levental, K. R., et al., 2015, Methods in Membrane Lipids, 1232:65-77; Scott, R. E., et al., 1976, Science, 194(4266):743-745; Sezgin, E., et al., 2012, Nature Protocols, 7(6):1042-1051). Mouse neuroblastoma cells (N2a) were used as a model cell line because of their ability to differentiate into neurons, making them suitable for studying synaptic transmission, neurotoxicity, and Alzheimer's disease (Khlistunova, I., et al., 2007, Current Alzheimer Research, 4(5):544-546; Cirrito, J. R., et al., 2008, Neuron, 58(1):42-51; Radio, N. M., et al., 2008, 29(3): 361-376). Fluorescently labeled N2a GPMVs were burst on the SLIM platform (FIG. 9B) and verified for lamellarity using α-hemolysin (FIG. 6C) Fluorescent micrographs showed the planar suspended lipid bilayer on the chip from GPMVs. The protocol was repeated for INS-1 insulinoma cells and Expi HEK293 suspension cells (FIG. 10) and can be applied to generate pore-spanning membranes from any cell type of interest. While N2a cells consistently gave bigger GPMVs (15.4 μm, FIG. 3E), GPMVs from INS-1 cells and HEK293 cells (9.4 μm and 8.9 μm, FIG. 10E) also formed bilayers. To verify the functionality, the diffusion coefficient of proteins from the native bilayers was measured, and values of 2.7±0.8 μm²/s for N2a cells, 2.8±0.4 μm²/s for INS-1 cells, and 2.1±0.5 μm²/s for ExpiHEK293 cells were obtained, consistent with the observations in the reconstituted systems.

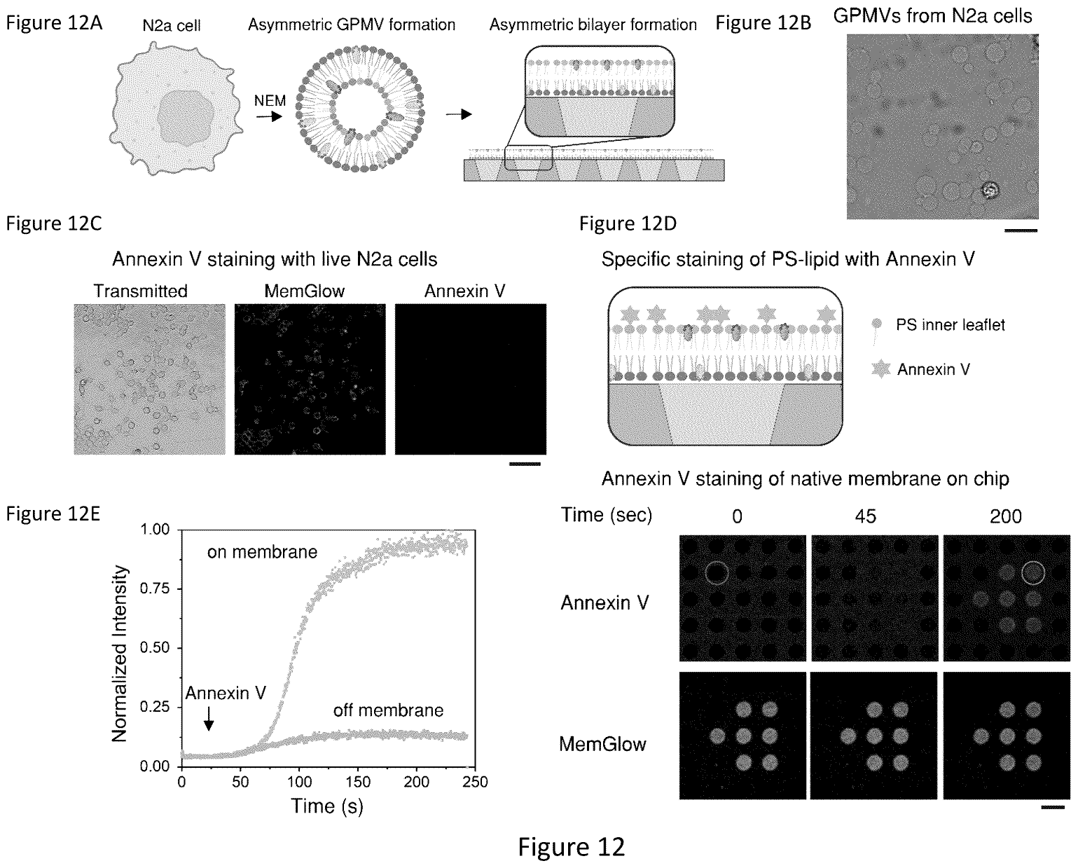

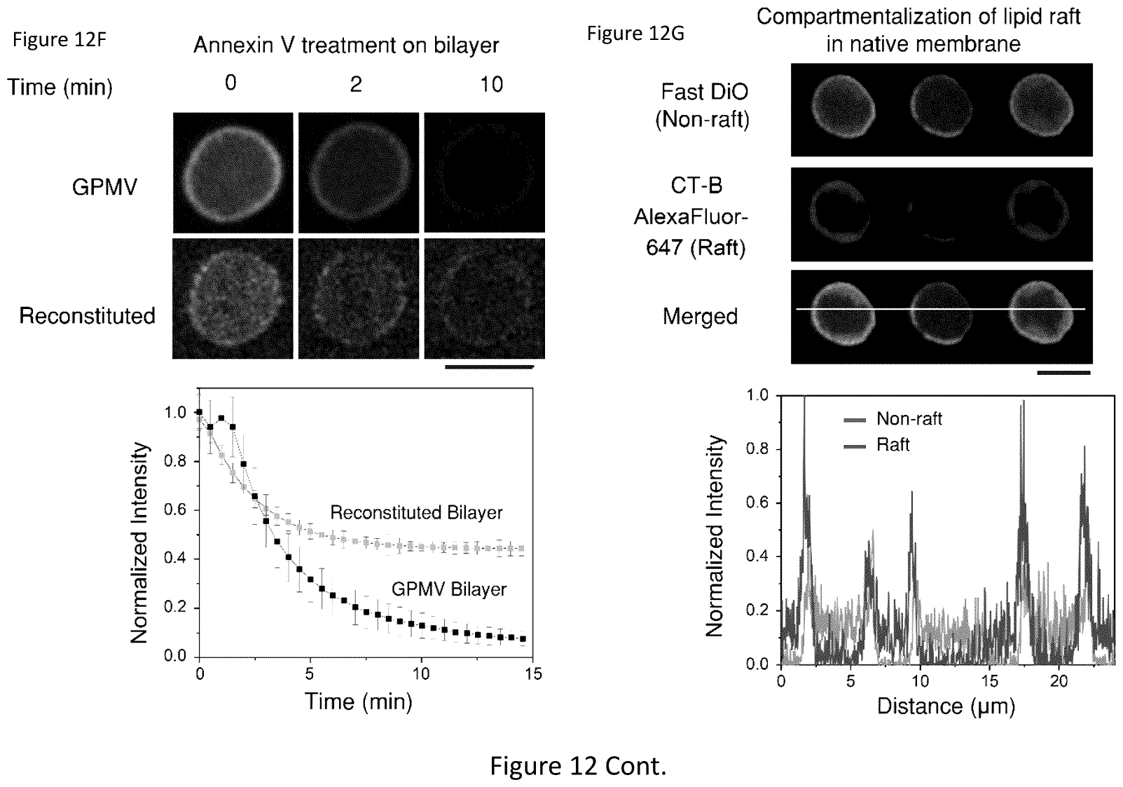

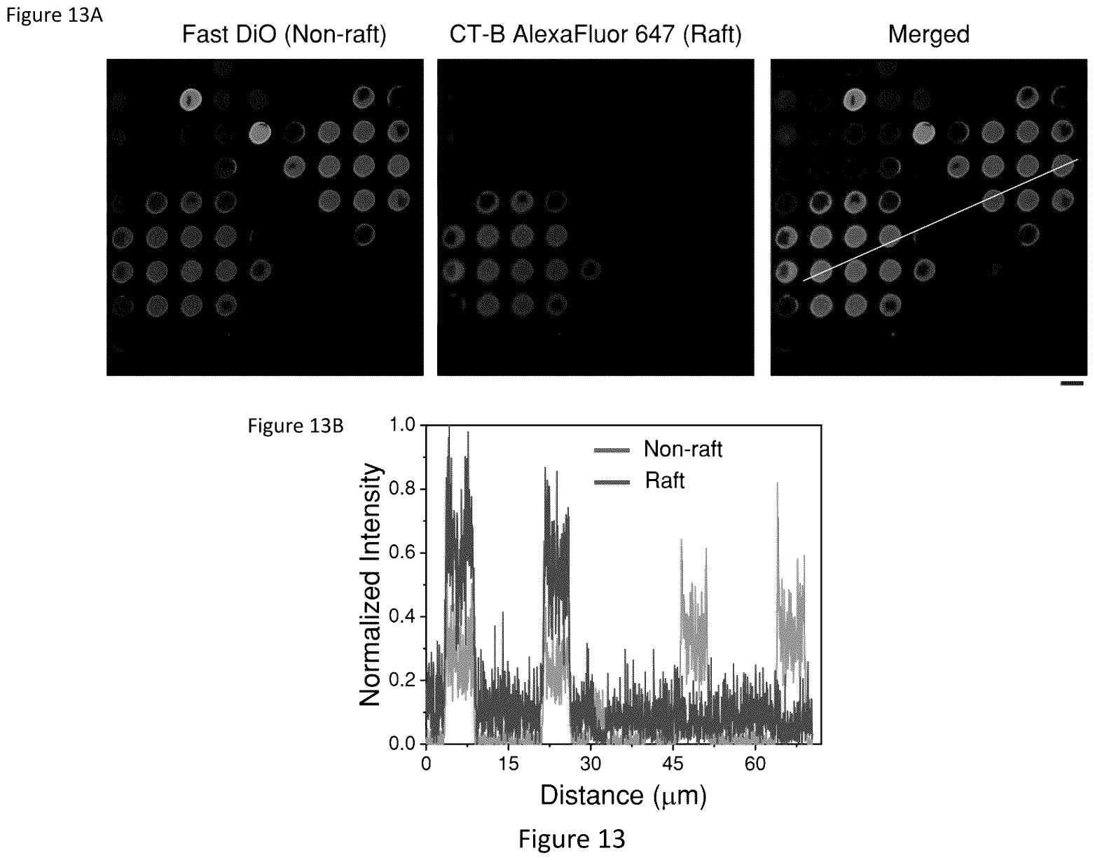

A challenge in protein reconstitution is the control over the directional insertion of proteins into lipid bilayers to mimic the cellular environment. Most reconstitutions lead to random protein orientations resulting in a significant fraction of the proteins not participating in the experiment. While some studies have attempted to control lipid asymmetry and protein orientation, the process is tedious, time-consuming, and difficult to replicate (Kamiya, K., et al., 2016, Nature Chemistry, 8(9):881-889; Hu, P. C., et al., 2011, ACS Applied Materials & Interfaces, 3(5): 1434-1440; Yanagisawa, M., et al., 2011, Journal of the American Chemical Society, 133(30): 11774-11779). This issue was avoided by making bilayers from cell GPMVs, which preserves the native direction of the proteins in the plasma membrane. After deposition, the GUVs spread on the chip, exposing the inner leaflet to the top. To confirm this, the membrane was tested for specific proteins found on the inner leaflet (SNAP25, Syntaxin1a) and outer leaflet (LRP-1) in N2a. Only SNAP25 and Syntaxin1a could be visualized on planar bilayers, while LRP-1 only showed up on intact cells and GPMVs, suggesting protein directionality was maintained (FIGS. 9F and 11). The proteins were observed as localized clusters, likely because these proteins are organized in PIP2 domains. Preservation of lipid direction was also examined. Phosphatidylserines are unique lipids that localize on the inner leaflet in healthy cells and flip outward when the cell is undergoing apoptosis. Using Annexin V, only the planar bilayers from the cells could be stained after bursting them on the SLIM platform suggesting PS remains on the inner leaflet (FIGS. 12C-12E). This was further verified when chymotrypsin treatment of cellular bilayers was compared with reconstituted membranes (FIG. 12F). The results show that only half the Annexin on the reconstituted membranes could be removed, but almost all Annexin could be removed from the cellular membranes, further verifying PS asymmetry being maintained. Next, raft and non-raft domains were examined using cholera toxin subunit B and Fast DiO staining, respectively. Distinct raft and non-raft domain staining patterns were observed within single bilayers (FIG. 12G) or distinct bilayers enriched in the raft and non-raft phases (FIG. 13). The procedure to harvest GPMVs and create bilayers preserves the protein orientation and native lipid architectures.

### Example 5: Ultra-High-Speed Imaging of Vesicular Exocytosis

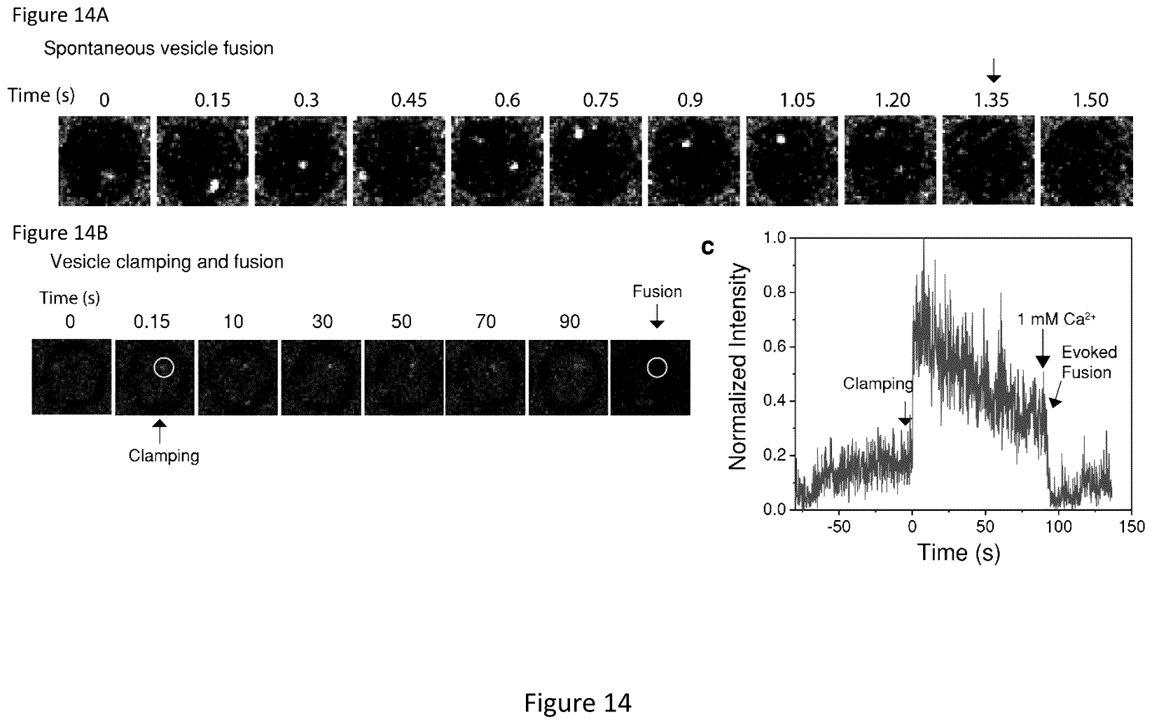

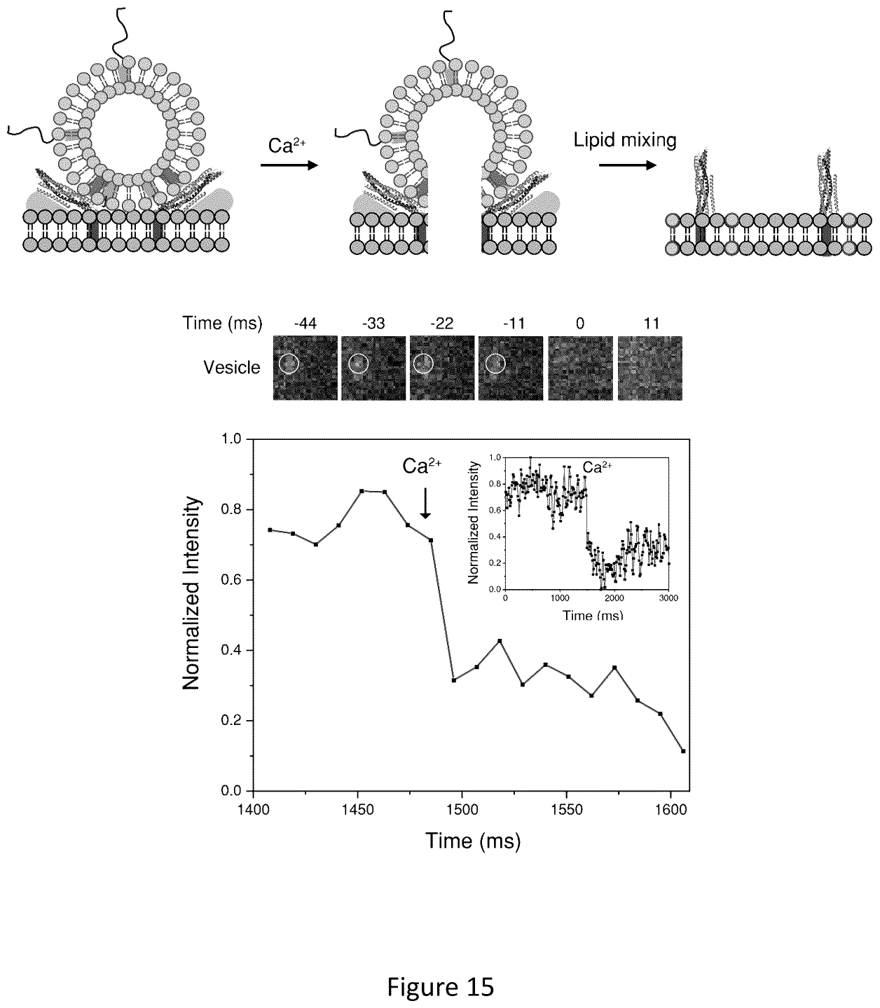

The SLIM system overcomes all the limitations of suspended and black lipid membranes with the ability to monitor dynamic biological processes with high signal-to-noise. Using this system, it was possible to monitor spontaneous fusion using labeled vesicles containing VAMP2 on the GPMV-based planar bilayers (FIG. 14) since they contain the essential t-SNARE proteins. To test evoked release, and build a readily releasable pool, vesicles containing reconstituted VAMP2, Synaptotagmin 1, and the soluble clamping protein Complexin 1, were used. The vesicles docked on the bilayer and didn't proceed to spontaneous fusion. Synchronized fusion of the vesicles was observed upon addition of calcium, showing that the system can be adapted to recreate physiological processes in vitro. FIG. 14C shows the fluorescence trace of the complete vesicle fusion process, including vesicle docking and fusion. The sudden drop in the fluorescence intensity is caused by vesicle fusion with the plasma membrane upon addition of calcium, resulting in the fluorescent lipids diffusing away from the vesicle ROI. The advantage of this system is that many events can be tracked simultaneously (FIG. 15) at high time-resolution, for a better understanding of any biological processes with higher quantitative statistics. On the reconstituted bilayers, vesicular exocytosis could be recorded at 89 frames per second (11 msec/frame). The protein copy numbers in the reconstituted vesicles were controlled to mimic physiology and their fusogenicity was independently verified by a bulk fusion assay (Takamori, S., et al., 2006, Cell, 127(4): 831-846; Scott, B. L., et al., 2003, Methods in Enzymology, 372:274-300). FIG. 14 shows the representative trace of evoked fusion between the t-SNARE bilayers and VSyt-SUVs.

Here, a suspended lipid membrane platform (SLIM) has been developed to perform single-molecule imaging experiments under in vitro conditions on reconstituted and native (cell-derived) lipid bilayers that retain cellular architecture To achieve this, a microarray design was engineered, and index-matching media fine-tuned to achieve the ability to see single molecules on the suspended bilayer. The chip design is useful for observing biological processes that happen in the millisecond time scale at the membrane interface with single-molecule resolution due to the high signal-to-noise ratio.

The SLIM platform has the potential for capturing transient membrane interactions due to its excellent maintenance of membrane properties, including lateral diffusion, molecular kinetics, and phase behavior. Experiments using fluorophore-conjugated DNA and proteins demonstrated the system has the ability to visualize single fluorescent molecules on suspended membranes. Subsequent experiments monitoring vesicle fusion confirm that lipids and proteins from cellular membranes are functional. Studies are ongoing to demonstrate that the system can be utilized for investigating ion channels and transport across membrane pores. OptiPrep™ (Iodixanol) is a relatively small molecule (1.5 kDa, 1.4 nm from edge-to-edge) known to not interact with biological molecules In tests on membrane bending, lipid diffusion, and protein diffusion it was found the differences between conditions with or without OptiPrep™ is insignificant. It was also found that OptiPrep™ does not block or clog α-hemolysin pores (2.8 nm pore size). Ongoing studies investigating molecular transport across channels are verifying that this is true with other channels.

A concern in producing GPMVs was the use of DTT or fixatives like PFA, which could render surface proteins inactive. In these experiments, it was demonstrated that N-ethyl maleimide treatment does not affect lipid mobility or protein mobility or function based on membrane fusion tests. The SLIM platform was engineered to provide easy access to the lipids and proteins on the inner leaflet of cells. Current methods using GPMVs derived from cells are not suitable for single-molecule and super-resolution imaging because they are intact or used to make supported bilayer with diffusion-related artifacts (Chiang, P.-C., et al., 2017, Scientific Reports, 7(1): 15139). The present method, however, integrates native or reconstituted bilayers in a planar, suspended format without losing lipid and protein functionality. Here it is demonstrated that the system, and the GPMV protocols, are robust and highly reproducible for both adherent (N2a and INS-1) and suspension (ExpiHEK293) cell types. The second notable feature of this system is that the inner leaflet of the cell membrane, and the corresponding proteins, are accessible for in vitro assays without damaging the membrane or requiring harsh fixing and permeabilizing protocols. It was validated that the native bilayers derived from cells preserve the protein and lipid asymmetry and function by imaging proteins (SNAP25, Syntaxin1, and LRP-1) and reconstituting single-vesicle fusion. It was also possible to recreate a readily releasable pool of vesicles that were sensitive to calcium-mediated release, suggesting retention of intact PIP2-rich domains, which would be present in the active zones of cell membranes (Honigmann, A., et al., 2013, Nature Structural and Molecular Biology, 20(6):679-686). The single-vesicle experiments demonstrate consistent behavior between native and reconstituted bilayers. Thus, the SLIM chip provides an ideal solution for in vitro membrane research, with the following advantages (1) solvent-free access to a suspended membrane in a planar geometry for single-molecule imaging, molecular counting, and FRET, (2) maintenance of lipid asymmetry and protein orientation, (3) preservation of protein diffusivity and function (4) reproducible suspension of planar bilayers from different cell types (N2a, INS-1, and Expi-HEK293) through optimized GPMV conditions, (5) temporal stability (3 hours) of suspended bilayer suitable for time-lapse imaging, and (6) functional membrane transport for cell signaling studies.

Previously reported cell-derived native bilayers have been mostly for lipid phase behavior studies and have not been applied to examine protein dynamics, functions, and membrane remodeling processes (Sezgin, E., et al., 2012, Nature Protocols, 7(6): 1042-1051). This is mainly because the circular geometry of the vesicles doesn't allow monitoring of proteins in microscopy On the other hand, the bilayer formed on the substrate doesn't permit membrane proteins to diffuse freely, limiting the applications (Pappa, A.-M., et al., 2020, ACS Nano, 14(10): 12538-12545). The SLIM platform helps overcome this problem by suspending bilayers over the holes of the platform. The behavior of the lipids and membrane proteins in the suspended bilayers resembles what is found in cells making this a good model system to work with. Although the supporting structures of the chip affect protein diffusion properties, each hole is big enough for membrane proteins to readily diffuse and interact with each other to perform biological processes for visualization by microscopy at single molecule resolution. This new method is highly suited to study lipid phase behavior, protein-protein interactions, and membrane dynamics. Ongoing studies are providing easy access to the outer leaflet, analysis by Raman microscopy, methods to filter GPMVs containing proteins of interest, and protocols to increase GPMV size and hence bilayer coverage.

The materials utilized and methods employed are described herein.

### Materials:

Lipids including 1,2-dioleoyl-sn-glycero-3-phosphocholine (DOPC), 1,2-dioleoyl-sn-glycero-3-phospho-L-serine) (DOPS) and phosphatidylinositol 4,5-bisphosphate (PIP2), 1,2-dioleoyl-sn-glycero-3-phosphoethanolamine-N-(7-nitro-2-1,3-benzoxadiazol-4-yl) (ammonium salt) (NBD-PE) were purchased from Avanti Polar Lipids (Alabaster, AL, USA). Fluorescent lipids (ATTO465-PE and ATTO647N-PE) were purchased from Attotec (Germany). For in situ membrane labeling, MemGlow™ 488 (MG01-02) and MemGlow™ 640 (MG04-02) were purchased from Cytoskeleton, Inc (Denver, CO). Maleimide conjugates (Alexa Fluor 568 and 647), Fluo-3, Pentapotassium Salt, F3715, Alexa Fluor 647 NHS Ester #A20006, Annexin V Alexa Fluor 488 conjugate #A13201, Fast DiO (3,3′-Dilinoleyloxacarbocyanine Perchlorate) #D3898 and CT-B Alexa Fluor 647 (cholera toxin subunit B) #C34778 were purchased from Thermofisher. Rabbit antibodies (anti-Syntaxin1A #A19243, anti-SNAP25 #A2234, and anti-LRP1 #A0633) were purchased from Abclonal. Secondary antibody goat-anti-Rabbit-Alexa647 conjugate #111-605-003 was purchased from Jackson Immuno Research Labs. Primers for single-molecule and FRET experiments were purchased from IDT Technologies (Coralville, IA). Neuro-2a mouse neuroblastoma cells were purchased from ATCC. ExpiHEK293 cells were purchased from Thermofisher. INS-1 (832/13) rat-derived insulinoma cells were obtained.

### Chip Fabrication:

The devices were fabricated on a 4-inch silicon-on-insulator (SOI) wafer with a device layer that had a silicon layer thickness of 4.7 μm and a 300 μm handle layer separated by a buried oxide layer. First, the SOI wafer was thermally oxidized to grow an oxide layer and then the defined hole diameter patterns created using lithography. The wells were etched into the silicon layer by reactive ion etching until the buried oxide layer. This was followed by anodically bonding a glass wafer on top of the etched wells. Finally, the handle silicon of the SOI wafer was removed by wet etching to create a final silicon chip (Micromotive, Germany). The chips were diced into 5×5 mm squares and glued to ibidi 8-well chambers for creation of a native plasma membrane on a chip.

### Single Molecule and FRET DNA Measurements:

For single-molecule step-bleaching experiments, primers containing biotin or fluorophores as shown below were ordered from IDT (Chatterjee, S., et al., 2012, Nanoscale, 4(5):1568-1571).

a)
(SEQ ID NO: 1)
5′-Biotin/TTT GGA TCC GCA TGA CAT TCG CCG TAA
G-3′
b)
(SEQ ID NO: 2)
5′-AlexaFluor 647N/CTT ACG GCG AAT GAC CGA ATC
AGC CT-3′
c)
(SEQ ID NO: 3)
5′-AGG CTG ATT CGG TTC ATG CGG ATC CA/AlexaFluor
647N-3′

For FRET measurements, DNA primers encoding the donor (internal iCy3) and acceptor (Cy5) were ordered from IDT as used previously (McCann, J. J., et al., 2010, Biophysical Journal, 99(3):961-970).

Donor:
(SEQ ID NO: 4)
5′-CGT GTC GTC GTG CGG CTC CCC AGG CG iCy3 G CAG
TCC-3′
Acceptor:
(SEQ ID NO: 5)
5′-Cy5-GGA CTG CCG CCT GGG GAG CCG CAC GAC
GAC ACG ACA AAG-TEG-Biotin-3′

Primers were initially solubilized in 1 mM TE buffer at 100 UM stock concentration. Primers were mixed to allow complex formation and then added to bilayers containing 1 mol % biotin-DOPE, pre-treated with 3% BSA and 800 nM neutravidin. After 30 minutes, the bilayer was washed and imaged at 569 nm (donor) and 651 nm (FRET). Videos were acquired at 20 fps (50 msec/frame). FRET imaging was done using DuoView2 to record donor and FRET emission channels simultaneously. Analysis was done using ImageJ and intensity plots generated from selected ROIs.

### Protein Production and Purification:

cDNA constructs expressing full length t-SNARE complex (mouse His6-SNAP25b and rat Syntaxin1a), full length v-SNARE protein (mouse VAMP2-His6) and Synaptotagmin (57-421, rat SYT1-5×His) were produced as previously described (Ramakrishnan, S., et al., 2020, eLife, 9:e54506; Kalyana Sundarama, R. V., et al., 2021, FEBS Letters, 595(3):297-309). Briefly, proteins were expressed in E. coli strain Rosetta 2 (DE3) (Novagen, Madison, WI) by inducing bacteria with 0.5 mM IPTG for 4 hours at 37° C. Cells were pelleted and lysed in buffer containing 400 mM KCl, 25 mM HEPES, 4% TritonX-100 (v/v), 10% glycerol (v/v), pH 7.4 supplemented with 0.2 mM Tris (2-carboxyethyl) phosphine hydrochloride (TCEP, ThermoFisher, Waltham, MA), and 1 mM phenylmethylsulfonyl fluoride (PMSF, SigmaAldrich). After running samples through the cell disruptor (Avestin, Ottawa, Canada), the lysate was clarified using a 45 Ti rotor (Beckman Coulter, Atlanta, GA) at 35 k rpm for 30 minutes at 4° C. The supernatant was then incubated with HisPur NiNTA beads (Thermofisher, Waltham, MA) with constant agitation overnight at 4° C. The resin was washed with lysis buffer containing 1% octyl glucoside (Chem-Impex, Wood Dale, IL). Protein was eluted with 350 mM imidazole and the concentration was determined using a Bradford Assay (BioRAD, Hercules, CA). SYT1 protein was further purified by running over an anion exchange column (MonoS, Cytiva) and eluted with an increasing ionic strength gradient. Protein was then aliquoted and flash-frozen with liquid nitrogen and stored at −80° C.

### Proteoliposome Reconstitution:

SNAREs used in this experiment have been modified to remove all Cys residues and add one at the N-terminus of the zippering domain C85G, C88G, C90G, C92G Q20C on SNAP25 and C103S, S28C on VAMP2. For the SYT1 counting experiment, SYT1 was modified to remove an endogenous cysteine (C277A) and add another at E269C. Proteins were labeled overnight with 5× excess of the dye in 1 mM TCEP. Excess dye was removed using dye removal columns (ThermoFisher, Waltham, MA). Lipid reconstitution was done as described previously (Ramakrishnan, S., et al., 2020, eLife, 9:e54506). For fusion assays, lipids used at 3 mM final in the v-reconstitution include 63% DOPC, 15% DOPS, 20% cholesterol and 2% ATTO647N-DOPE. For smFRET assays, the ATTO647N-DOPE was replaced with DOPC. Lipids were dried under nitrogen followed by vacuum in glass vials. VAMP2 and SYT1 proteins were mixed with lipids (1:100 and 1:225 respectively) in buffer containing 1% OG. For smFRET, VAMP2 (1:556) was used and SYT1 was left out. After shaking on a vortex for 30 minutes, the v-SNARE samples were diluted three times in buffer without OG. Samples were dialyzed overnight to remove detergent and floated up in Nycodenz gradients using an SW55 rotor (BeckmanCoulter) for 4 hours at 48,000 rpm at 4° C.

### Creating GPMVs:

N2a cells were cultured with Dulbecco's Modified Eagle's Medium (DMEM) supplemented with 10% fetal bovine serum and 100 μg/mL penicillin/streptomycin (Gibco) in 5% CO₂ at 37° ° C. in a humidified incubator. Cells were 95% viable and more than 80% confluent before being treated with NEM. Cells were washed twice with PBS and incubated with MemGlow™ 488 or 647 lipophilic probe at 4° C. for 10 minutes to allow incorporation of MemGlow™ dye into the cell plasma membranes. Prior to GPMV isolation, cells were washed twice with PBS and vesiculating buffer. Cells were incubated with 1 ml of vesiculating buffer containing 2 mM of N-ethyl maleimide (NEM), 10 mM HEPES, 150 mM KCl, 2 mM CaCl₂), 10 mM Sucrose, pH 7.4 for 1 hour at 37° C. The GPMV-rich cellular supernatant was transferred into a microcentrifuge tube using a cut pipette before being used to form suspended lipid bilayers. For imaging experiments, GPMVs were transferred to Mattek dishes coated with 3% BSA for 30 minutes prior to addition to prevent GPMVs from forming supported bilayers.

### Labeling Cells, GPMVs and GPMV-Derived Bilayers:

To monitor the formation of GPMVs and track the plasma membrane dynamics, MemGlow™ 488 or 640 was used to label the lipid bilayer. Cells were grown in Mattek dishes 24 hours prior to the experiment. The cultured cells were first washed with PBS and stained with 20 nM of MemGlow™ solution for 30 min at 4° C. in a dark environment. The cells were then washed two more times with GPMV buffer to remove any unincorporated dye. Finally, 1 mL of GPMV solution with 2 mM of NEM was added to form GPMVs and monitored the GPMVs formation at 37° C. under microscope settings. For antibody staining, primary antibodies were used at 1:1000 dilution for 1 hour. Cells were washed gently with PBS three times and then stained with secondary antibody for 30 minutes. Cells were washed in PBS three times prior to imaging. For lipid asymmetry and domain detection, we stained with Annexin V. Fast DiO and CT-B for 10 minutes before washing ten times with GPMV buffer. In the experiments with chymotrypsin treatment, the protease was added after the wash and observed for 15 minutes with images were taken every 30 seconds to mitigate photobleaching effects For measuring protein diffusion on native bilayers, all proteins were labeled using NHS ester conjugated to AlexaFluor 647 for 45 minutes. Samples were washed ten times with PBS and then imaged. Protein FRAP measurements were done at 147 ms/frame. An elliptical ROI covering 25% of the bilayer was used. The sample was bleached for 300 frames at 100% laser power. The fluorescence intensity during bleaching was fit with an exponential curve to determine the characteristic time and diffusion constant.

### Live-Cell Imaging:

For cellular imaging, cells were plated on 35 mm tissue culture dishes with a cover glass bottom (refractive index=1.51; MatTek Life Sciences), Measurements were performed with a Leica SP8 Laser Scanning confocal microscope equipped with two PMTs, three lasers for excitation, and a 60× oil objective (NA 1.49), which produced a 1 μm-thick optical slice Cell imaging was performed in vesiculating buffer with anti-SNAP25, STX1A, and LRP antibodies.

### Suspended Lipid Bilayer Formation:

Suspended bilayers were made with either reconstituted lipid membranes or GPMVs harvested from cells. For the reconstituted bilayers, lipids were reconstituted at 15 mM, made up of 66.9 mol % DOPC, 30 mol % DOPS, 3 mol % PIP2, and 0.1 mol % ATTO465-PE. After drying under nitrogen followed by vacuum, lipids were rehydrated in a high salt buffer (600 mM KCl, 125 mM HEPES, 1 mM TCEP, pH 7.4) containing protein (t-SNARE) to lipid ratio of 1:800 and 1% octyl-glucoside (OG). For FRET experiments, t-SNARE proteins were diluted to 1:10,000 (P:L). The detergent was dialyzed away overnight in buffer without detergent. The dialyzed sample was air-dried twice on a clean glass slide after being supplemented with 0.4 mM sucrose. The sample was finally rehydrated in a volume five times the original drop size to form GUVs (Motta, I., et al., 2015, Langmuir, 31(25): 7091-7099). The final buffer solution of GUV is 25 mM, 120 mM KCl, and 0.2 mM TCEP, which is the same as the buffer in the chip. Fluorescent lipids were left out of the lipid mix whenever fluorescently labeled proteins were reconstituted

Si/SiO₂ chips on the SLIM platform were plasma cleaned and rinsed with ethanol and water prior to use. Buffer [25 mM HEPES, 120 mM KCl, 0.2 mM TCEP, pH 7.4] supplemented with 45% OptiPrep™ and 5 mM MgCl₂ was then added to the chip. To ensure it got into the holes, the chip was then placed in low vacuum conditions which helped displace air bubbles that collected within the arrays with OptiPrep™ supplemented buffer. The GUVs or harvested GPMVs were transferred onto the chips. The lipids were incubated for 20 minutes before being centrifuged at 100×g to mediate the attachment and rupture of GUVs on the chip. The incorporated sucrose helps the GUVs attach to the silicon chip and rupture. It was found that the time-dependent, controlled formation of GUVs (˜45 mins) and rupture (˜30 mins) protocol is crucial for lipid bilayer spreading on the chip. After bilayer formation, the OptiPrep™ solution above the lipid bilayer was replaced with buffer supplemented with 1 mM MgCl₂ by pipetting 300 μL of buffer, three times using a P1000 pipette. The chip was mounted for imaging on a heated stage (iBidi).

### Single Vesicle Assay:

The function of lipids and proteins in our suspended bilayer was demonstrated by reproducing the fundamental biological process, exocytosis, in an in vitro environment by previously well-established single vesicle fusion assays (Ramakrishnan, S., et al., 2018, Langmuir, 34(20):5849-5859; Bera, M., et al., 2022, eLife, 11:e71938; Coleman, J., et al., 2018, 22(3): 820-831; Ramakrishnan, S., et al., 2019, FEBS Letters, 593(2): 154-162; Ramakrishnan, S., et al., 2020, eLife, 9:e54506). Briefly, ATTO 647N fluorophore-labeled reconstituted vesicles containing 60 copies of VAMP2 and 20 copies of SYT1 were added to the chip containing t-SNARE membranes derived from cells or reconstituted and allowed to monitor the fate of vesicles stages including vesicle docking, clamping, and fusion. A Nikon inverted microscope equipped with 3 laser lines (488, 532, and 633 nm), a Photometrics DV2 dual view, and an Andor EMCCD digital camera was used to monitor the fate of vesicles at 11 milliseconds/frame rate. Two fluorophore channels, bilayer, and vesicle were simultaneously imaged to monitor lipid vesicle fusion. To reconstitute calcium-mediated exocytosis, we pre-incubated the bilayer with 2 μM CPX and monitored the vesicle fusion by the addition of 1 mM CaCl₂).

### Trapped Fluorophore Experiments:

20 mM soluble sulforhodamine B (SRB, S1307 Thermo Fisher) was mixed in the buffer before bilayer formation. After bursting the GUV on the chip, using 5 mM MgCl₂, the excess the SRB dye from the chamber was completely replaced with buffer containing 25 mM HEPES, 120 mM KCl, pH 7.4, 1 mM DTT. Then 20% detergent solution (sodium dodecyl sulfate, SDS) was added to solubilize the lipid membrane to release the captured fluorophores. Similarly, for calcium trapping, 25 mM calcium chloride (Millipore Sigma 10043-52-4) was added to the platform before GUV bursting. After bilayer formation, the buffer was replaced with 10 mM of calcium indicator (Fluo-3, Penta potassium Salt, #F3715). Finally, 0.5 mM α-hemolysin (Millipore Sigma, #94716-94-6) was added into the chamber and the intensity monitored the intensity.

### Chymotrypsin Treatment:

2 mM chymotrypsin (Millipore Sigma #9004-07-3) was mixed with proteo-liposomes and incubated for 30 minutes at 37° C. The reaction was quenched with the addition of 10 mM HCl. For imaging experiments, samples were floated in a Nycodenz® gradient and visualized on a clean glass slide. Samples were also run on SDS-PAGE gel. Only the inward facing proteins that were protected from chymotrypsin were stained with Coomassie blue. The gel band intensities were calculated using ImageJ software.

### Determination of Requirements for TIRF:

To achieve total internal reflection at the bilayer, an intermediate index-matching medium (n₂) was added between the glass (n₁) and bilayer (n₃). In generic TIRF microscopy, only two refractive indexes (RI) are involved. The fluorescence excitation beam enters the cover glass (n₁) at a critical angle, θc, into a lower refractive index of the buffer (n₂), creating total internal reflection at the boundary. The incident excitation beam is completely reflected off at the glass-buffer interface and only an evanescent wave penetrates beyond it, decaying exponentially with the distance from the boundary. A medium with an RI greater than the buffer was utilized to get the evanescent wave at the bilayer, which is 4.7 μm above the glass coverslip. Towards this goal, sucrose and Iodixanol (OptiPrep™) were tested to create a refractive index greater than 1.4. Sucrose (˜50% solution) results in a substantial osmolarity difference between the index-matching medium and the physiological buffer, resulting in rupture of the suspended lipid membranes, making the reagent incompatible. OptiPrep™, on the other hand, displayed an osmolality of 260±2 mmol/kg at 60% stock solution with a RI of 1.43, which allows maintenance of bilayer stability (Graham, J., et al., 1994, Analytical Biochemistry, 2020:367-373). The solubility and refractive index of Iodixanol were tested in distilled water and buffer. A serial dilution was used to linearly tune the refractive index of the solutions between 1.333-1.429 (FIG. 4A). 45% OptiPrep™ solution was used as an index matching medium, with an RI of 1.40-1.41, to produce TIR at the OptiPrep™-bilayer interface. In this configuration, the incident beam crosses the interface between the glass and the OptiPrep™, such that θ_{I}<θ_{C}=arcsin (n_{g}/n_{OptiPrep}™). Rays with θ_{I} greater than the critical angle θ_{C} for the glass-OptiPrep™ interface will result in total internal reflection and not enter the OptiPrep™ index-matching medium, thus contributing to the reflection at the glass-OptiPrep™ boundary rather than the TIRF signal.

$${θ}_{critical} = {sin}^{- 1} \frac{{n}_{water}}{{n}_{{OptiPrep}^{TM}}}$$

Where n_{water} is the refractive index of the medium that light is passing into and n_{OptiPrep}=the refractive index of the medium that light is passing out of. Such a setup allows the evanescent field to reach the bilayer-buffer interface by setting up the incident light to enter the glass at 62°, will refract the light beam to 72º, which is above the critical angle θc≥70° (OptiPrep™ (n₂)=1.41 and buffer (n₃)=1.33) at the bilayer

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A membrane on a chip device comprising:
a bottom layer of an essentially impermeable substrate having a first thickness;
a chip layer of an essentially impermeable substrate having a second thickness stacked on top of the bottom layer; and
a barrier layer of an essentially impermeable material having a third thickness stacked on top of the chip layer;
wherein the barrier layer comprises one or more macro-holes extending through the thickness of the barrier layer with a first diameter, exposing the chip layer; and
wherein the chip layer comprises a plurality of holes extending through the thickness of the barrier layer with a second diameter, wherein the plurality of holes is arranged in a number of rows and columns with a regular distance separating the holes of each row and each column.

2. The chip of claim 1, wherein the first layer comprises SF11 glass, soda-lime glass, borosilicate glass, aluminosilicate glass, or glass-ceramic and has a thickness of about 0.05 mm to about 5 mm.

3. The chip of claim 2, wherein the thickness is about 150 μm.

4. The chip of claim 1, wherein the second layer comprises Si/SiO₂ or polydimethylsiloxane (PDMS) and has a thickness of about 2.5 μm to about 6 μm.

5. The chip of claim 1, wherein the holes are cylindrical and have a diameter of about 1-7.5 μm.

6. The chip of claim 5, wherein the holes are right circular frustra with a lower diameter greater than the upper diameter, wherein the upper diameter is about 1 μm to about 10 μm and the lower diameter is about 1.1-15.4 μm.

7. The chip of claim 5, wherein the distance between holes in the rows and the distance between holes in the columns is about half the diameter of the holes.

8. The chip of claim 4, wherein the thickness of the second layer is 4.7 μm, the holes are cylindrical, the diameter of the holes is 5 μm, and the distance between the holes is 2.5 μm.

9. The chip of claim 4, wherein the thickness of the second layer is 4.7 μm, the holes are frustra with an upper diameter of about 5 μm and a lower diameter of about 14.1 μm, and the distance between the holes is 5 μm.

10. The chip of claim 1, wherein the holes of a row are connected by a series of channels.

11. The chip of claim 10, wherein the first and last holes are connected to openings on the exterior edge of the second layer by channels such that a fluid could flow from the opening connected to the first hole, through the channels and holes, out the opening connected to the last hole.

12. The chip of claim 1, wherein the holes comprise electrodes operably linked to wires which extend through the first layer of the chip.

13. A system for studying membranes comprising a microscope,
spectrometer, or spectrophotometer and a membrane on a chip device, wherein the chip comprises:
a bottom layer of an essentially impermeable substrate having a first thickness;
a chip layer of an essentially impermeable substrate having a second thickness stacked on top of the bottom layer; and
a barrier layer of an essentially impermeable material having a third thickness stacked on top of the chip layer;
wherein the barrier layer comprises one or more macro-holes extending through the thickness of the barrier layer with a first diameter, exposing the chip layer; and
wherein the chip layer comprises a plurality of holes extending through the thickness of the barrier layer with a second diameter, wherein the plurality of holes is arranged in a number of rows and columns with a regular distance separating the holes of each row and each column.

14. The system of claim 13, wherein the microscope is a total internal fluorescence (TIRF) microscope.

15. The system of claim 13, wherein the spectrometer is a Raman spectrometer.

16. The system of claim 13, wherein the membrane contains proteins with specific directionality and symmetric or asymmetric distribution of lipids between leaflets.

17. The system of claim 13, wherein the membranes are reconstituted or are harvested from cells or parts of live or fixed cells.

18. The system of claim 13, wherein the membrane is suspended in the system such that it is compatible with single-molecule studies.

19. A kit comprising a membrane on a chip device, wherein the chip comprises:
a bottom layer of an essentially impermeable substrate having a first thickness;
a chip layer of an essentially impermeable substrate having a second thickness stacked on top of the bottom layer; and
a barrier layer of an essentially impermeable material having a third thickness stacked on top of the chip layer;
wherein the barrier layer comprises one or more macro-holes extending through the thickness of the barrier layer with a first diameter, exposing the chip layer; and
wherein the chip layer comprises a plurality of holes extending through the thickness of the barrier layer with a second diameter, wherein the plurality of holes is arranged in a number of rows and columns with a regular distance separating the holes of each row and each column.

20. The kit of claim 19, wherein the kit further comprises vesicles for generating a unilamellar plasma membrane on the chip and/or buffers and solutions including the index matching medium and/or a stabilization agent and/or instructional material.
